SEQUENCE LISTING (11 sequences)

SEQ ID NO: 1 - protein, 728 aa, Homo sapiens
MWVTKLLPALLLQHVLLHLLLLPIAIPYAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLRVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

SEQ ID NO: 2 - protein, 723 aa, Homo sapiens
MWVTKLLPALLLQHVLLHLLLLPIAIPYAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLRVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

SEQ ID NO: 3 - DNA, 7113 bp, Artificial Sequence
  Description of Artificial Sequence Synthetic polynucleotide
atgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacaggtaagaacagtatgaagaaaagagatgaagcctctgtcttttttacatgttaacagtctcatattagtccttcagaataattctacaatcctaaaataacttagccaacttgctgaattgtattacggcaaggtttatatgaattcatgactgatatttagcaaatgattaattaatatgttaataaaatgtagccaaaacaatatcttaccttaatgcctcaatttgtagatctcggtatttgtgaaataataacgtaaacttcgtttaaaaggattcttcttcctgtctttgagaaagtacggcactgtgcagggggagaggttgattgtgaaaaatcagaggtagatgagaatcttactgagggctgagggttctttaaccttggtggatctcaacattggttgcacattaaaatcacctgctgcaagcccttgacgaatcttacttagaagatgacaacacagaacaattaaatcagaatctctggggagaatagggcaccagtattttttgagctcccaccatgattccaaagtgcagccaaatttgagaaccactgctaaaagctcaagcttcagattgaccagcttttccatctcacctatcgcctaaagaccaaattggataaatgtgttcattacgacagatgggtactatttaaagatgagtaaacacaatatacttaggctcgtcagactgagagttttaatcatcactgaggaaaaacatagatatctaatactgactggagtattagtcaaggcttatttcacacacaattttatcagaaaccaaagtagtttaaaacagctctccccttattagtaatgcattggagggtttactttaccatgtaccttgctgagcactgtaccttgttaatctcatttacttgtaatgagaaccacacagcgggtagttttattggttctattttacctacatgacaaaactgaagcataaaaacacttagtaagttttcagtgtcatgcacaactaggaagtgacatggccagaatataagcccagtcaccatcactctataacctgcgcttttaacaacttcagggcatgacacatttggccggtcagtagaacccatgctgtgatttgtttttgcagtggtggtgatgactgccttgttgaatccactttttattctattccattttggggacacaattctgcaagatgattcttcattaggaaacagagatgagttattgaccaacacagaaagaaaaagagtttgttgctccacactgggattaaacctatgatcttggcctaattaacactagctagtaagtgtccaagctgatcatctctacaacatttcaataacagaaaacaacaattttcaaaattagttacttacaattatgtagaaatgcctctaaaacacagtattttccttatattacaaaaacaaaaattataattggttttgtcctcttttgagagtttgcatggtgttactccctgcatagtgaagaaaacattttatttaagtagatggatctaagtttttcatgaacaaaggaatgacatttgaaatcaatcctaccctagtccaggagaatgcattagattaacctagtagaggtcttatttcaccctgagttttctatgatcgtgattctctgctggaggagtaattgtgaaatagatctctctgggaactggcttcctagtccaatcagctcttttaccaatgaacacttccttgtgatatagatgtttatggccgagaggatccagtatattaataaaatccctttttgtattcaatgagggaaacacataattttcatcaattagcagcttattggaatatctgcatgatggtttaacacttttaagtgttgactaaagattaattttacagaaaatagaaaaagaaatatgtttctgtctggaggaatgatttattgttgacccctaaattgaaatattttactagtggcttaatggaaagatgatgaaagatgatgaaattaatgtagaagcttaactagaaaatcaggtgacctgatatctacatctgtatccttcattggccacccagcattcattaatgaatcagatgatggaatagatcaagtttcctaggaacacagtgaatattaaaagaaaacaaagggagcctagcacctagaagacctagtttatatttcaaagtatatttggatgtaacccaattttaaacatttcctcacttgtctctcttaaagccttgccaacagcaaggacagagaaccaaaaatagtgtatatatgaataaatgcttattacagaatctgctgactggcacatgctttgtgtgtaatgggttctcataaacacttgttgaatgaacacacataagtgaaagagcatggctaggcttcatcccttggtcaaatatggggtgctaaagaaaagcaggggaaatacattgggacactaacaaaaaaaaacagttaatttaggtaaaagataaaatacaccacagaatgaagaaaagagatgacccagactgctctttaaccttcatgtcctagagaggtttttgatatgaattgcattcagaattgtggaaaggagcccatcttttctcttcattttgattttattaactccaatgggggaattttattcgtgttttggccatatctacttttgatttctacattattctctcttcctttctacctgtatttgtcctaataaattgttgacttattaattcactacttcctcacagcttttttttggctttacaaatccactggaaaggtatatgggtgtatcactttgtgtatttcggtgtgcatgtgtagaggggacaaaaatcctctctcaaactataaatattgagtatttgtgtattgaacatttgctataactactaggtttcttaaataatcttaatatataaaatgatatagaaaaagggaaattatagttcgtattattcatctaagtgaagagattaaaacccagggagtaaataaattgtctaaggactaaggttgtatactatttaggtgatagatatggggcaaccgtatgggttttatgattaacaaataaacttctcaccactctaccatatcaacttttccataaaagagagctatagtattctttgcttaaataaatttgattagtgcatgacttcttgaaaacatataaagcaaaagtcacatttgattctatcagaaaagtgagtaagccatggcccaaacaaaagatgcattaaaatattctggaatgatggagctaaaagtaagaaaaatgactttttaaaaaagtttactgttaggaattgtgaaattatgctgaattttagttgcattataatttttgtcagtcatacggtctgacaacctgtcttatttctatttccccatatgaggaatgctagttaagtatggatattaactattactacttagatgcattgaagttgcataatatggataatacttcactggttccctgaaaatgtttagttagtaataagtctcttacactatttgttttgtccaataatttatattttctgaagacttaactctagaatacactcatgtcaaaatgaaagaatttcattgcaaaatattgcttggtacatgacgcatacctgtatttgttttgtgtcacaacatgaaaaatgatggtttattagaagtttcattgggtaggaaacacatttgaatggtatttactaagatactaaaatccttggacttcactctaattttagtgccatttagaactcaaggtctcagtaaaagtagaaataaagcctgttaacaaaacacaaactgaatattaaaaatgtaactggattttcaaagaaatgtttactggtattacctgtagatgtatattctttattatgatcttttgtgtaaagtctggcagacaaatgcaatatctaattgttgagtccaatatcacaagcagtacaaaagtataaaaaagacttggccttttctaatgtgttaaaatactttatgctggtaataacactaagagtagggcactagaaattttaagtgaagataatgtgttgcagttactgcactcaatggcttactattataaaccaaaactgggatcactaagctccagtcagtcaaaatgatcaaaattattgaagagaataagcaattctgttctttattaggacacagtagatacagactacaaagtggagtgtgcttaataagaggtagcatttgttaagtgtcaattactctattatcccttggagcttctcaaaataaccatataaggtgtaagatgttaaaggttatggttacactcagtgcacaggtaagctaataggctgagagaagctaaattacttactggggtctcacagtaagaaagtgagctgaagtttcagcccagatttaactggattctgggctctttattcatgttacttcatgaatctgtttctcaattgtgcagaaaaaagggggctatttataagaaaagcaataaacaaacaagtaatgatctcaaataagtaatgcaagaaatagtgagatttcaaaatcagtggcagcgatttctcagttctgtcctaagtggccttgctcaatcacctgctatcttttagtggagctttgaaattatgtttcagacaacttcgattcagttctagaatgtttgactcagcaaattcacaggctcatctttctaacttgatggtgaatatggaaattcagctaaatggatgttaataaaattcaaacgttttaaggacagatggaaatgacagaattttaaggtaaaatatatgaaggaatataagataaaggatttttctaccttcagcaaaaacatacccactaattagtaaaattaataggcgaaaaaaagttgcatgctcttatactgtaatgattatcattttaaaactagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatag

SEQ ID NO: 4 - DNA, 6190 bp, Artificial Sequence
  Description of Artificial Sequence Synthetic polynucleotide
atgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacaggtaagaacagtatgaagaaaagagatgaagcctctgtcttttttacatgttaacagtctcatattagtccttcagaataattctacaatcctaaaataacttagccaacttgctgaattgtattacggcaaggtttatatgaattcatgactgatatttagcaaatgattaattaatatgttaataaaatgtagccaaaacaatatcttaccttaatgcctcaatttgtagatctcggtatttgtgaaataataacgtaaacttcgtttaaaaggattcttcttcctgtctttgagaaagtacggcactgtgcagggggagaggttgattgtgaaaaatcagaggtagatgagaatcttactgagggctgagggttctttaaccttggtggatctcaacattggttgcacattaaaatcacctgctgcaagcccttgacgaatcttacttagaagatgacaacacagaacaattaaatcagaatctctggggagaatagggcaccagtattttttgagctcccaccatgattccaaagtgcagccaaatttgagaaccactgctaaaagctcaagcttcagattgaccagcttttccatctcacctatcgcctaaagaccaaattggataaatgtgttcattacgacagatgggtactatttaaagatgagtaaacacaatatacttaggctcgtcagactgagagttttaatcatcactgaggaaaaacatagatatctaatactgactggagtattagtcaaggcttatttcacacacaattttatcagaaaccaaagtagtttaaaacagctctccccttattagtaatgcattggagggtttactttaccatgtaccttgctgagcactgtaccttgttaatctcatttacttgtaatgagaaccacacagcgggtagttttattggttctattttacctacatgacaaaactgaagcataaaaacacttagtaagttttcagtgtcatgcacaactaggaagtgacatggccagaatataagcccagtcaccatcactctataacctgcgcttttaacaacttcagggcatgacacatttggccggtcagtagaacccatgctgtgatttgtttttgcagtggtggtgatgactgccttgttgaatccactttttattctattccattttggggacacaattctgcaagatgattcttcattaggaaacagagatgagttattgaccaacacagaaagaaaaagagtttgttgctccacactgggattaaacctatgatcttggcctaattaacactagctagtaagtgtccaagctgatcatctctacaacatttcaataacagaaaacaacaattttcaaaattagttacttacaattatgtagaaatgcctctaaaacacagtattttccttatattacaaaaacaaaaattataattggttttgtcctcttttgagagtttgcatggtgttactccctgcatagtgaagaaaacattttatttaagtagatggatctaagtttttcatgaacaaaggaatgacatttgaaatcaatcctaccctagtccaggagaatgcattagattaacctagtagaggtcttatttcaccctgagttttctatgatcgtgattctctgctggaggagtaattgtgaaatagatctctctgggaactggcttcctagtccaatcagctcttttaccaatgaacacttccttgtgatatagatgtttatggccgagaggatcccttcctttctacctgtatttgtcctaataaattgttgacttattaattcactacttcctcacagcttttttttggctttacaaatccactggaaaggtatatgggtgtatcactttgtgtatttcggtgtgcatgtgtagaggggacaaaaatcctctctcaaactataaatattgagtatttgtgtattgaacatttgctataactactaggtttcttaaataatcttaatatataaaatgatatagaaaaagggaaattatagttcgtattattcatctaagtgaagagattaaaacccagggagtaaataaattgtctaaggactaaggttgtatactatttaggtgatagatatggggcaaccgtatgggttttatgattaacaaataaacttctcaccactctaccatatcaacttttccataaaagagagctatagtattctttgcttaaataaatttgattagtgcatgacttcttgaaaacatataaagcaaaagtcacatttgattctatcagaaaagtgagtaagccatggcccaaacaaaagatgcattaaaatattctggaatgatggagctaaaagtaagaaaaatgactttttaaaaaagtttactgttaggaattgtgaaattatgctgaattttagttgcattataatttttgtcagtcatacggtctgacaacctgtcttatttctatttccccatatgaggaatgctagttaagtatggatattaactattactacttagatgcattgaagttgcataatatggataatacttcactggttccctgaaaatgtttagttagtaataagtctcttacactatttgttttgtccaataatttatattttctgaagacttaactctagaatacactcatgtcaaaatgaaagaatttcattgcaaaatattgcttggtacatgacgcatacctgtatttgttttgtgtcacaacatgaaaaatgatggtttattagaagtttcattgggtaggaaacacatttgaatggtatttactaagatactaaaatccttggacttcactctaattttagtgccatttagaactcaaggtctcagtaaaagtagaaataaagcctgttaacaaaacacaaactgaatattaaaaatgtaactggattttcaaagaaatgtttactggtattacctgtagatgtatattctttattatgatcttttgtgtaaagtctggcagacaaatgcaatatctaattgttgagtccaatatcacaagcagtacaaaagtataaaaaagacttggccttttctaatgtgttaaaatactttatgctggtaataacactaagagtagggcactagaaattttaagtgaagataatgtgttgcagttactgcactcaatggcttactattataaaccaaaactgggatcactaagctccagtcagtcaaaatgatcaaaattattgaagagaataagcaattctgttctttattaggacacagtagatacagactacaaagtggagtgtgcttaataagaggtagcatttgttaagtgtcaattactctattatcccttggagcttctcaaaataaccatataaggtgtaagatgttaaaggttatggttacactcagtgcacaggtaagctaataggctgagagaagctaaattacttactggggtctcacagtaagaaagtgagctgaagtttcagcccagatttaactggattctgggctctttattcatgttacttcatgaatctgtttctcaattgtgcagaaaaaagggggctatttataagaaaagcaataaacaaacaagtaatgatctcaaataagtaatgcaagaaatagtgagatttcaaaatcagtggcagcgatttctcagttctgtcctaagtggccttgctcaatcacctgctatcttttagtggagctttgaaattatgtttcagacaacttcgattcagttctagaatgtttgactcagcaaattcacaggctcatctttctaacttgatggtgaatatggaaattcagctaaatggatgttaataaaattcaaacgttttaaggacagatggaaatgacagaattttaaggtaaaatatatgaaggaatataagataaaggatttttctaccttcagcaaaaacatacccactaattagtaaaattaataggcgaaaaaaagttgcatgctcttatactgtaatgattatcattttaaaactagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatag

SEQ ID NO: 5 - DNA, 5190 bp, Artificial Sequence
  Description of Artificial Sequence Synthetic polynucleotide
atgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacaggtaagaacagtatgaagaaaagagatgaagcctctgtcttttttacatgttaacagtctcatattagtccttcagaataattctacaatcctaaaataacttagccaacttgctgaattgtattacggcaaggtttatatgaattcatgactgatatttagcaaatgattaattaatatgttaataaaatgtagccaaaacaatatcttaccttaatgcctcaatttgtagatctcggtatttgtgaaataataacgtaaacttcgtttaaaaggattcttcttcctgtctttgagaaagtacggcactgtgcagggggagaggttgattgtgaaaaatcagaggtagatgagaatcttactgagggctgagggttctttaaccttggtggatctcaacattggttgcacattaaaatcacctgctgcaagcccttgacgaatcttacttagaagatgacaacacagaacaattaaatcagaatctctggggagaatagggcaccagtattttttgagctcccaccatgattccaaagtgcagccaaatttgagaaccactgctaaaagctcaagcttcagattgaccagcttttccatctcacctatcgcctaaagaccaaattggataaatgtgttcattacgacagatgggtactatttaaagatgagtaaacacaatatacttaggctcgtcagactgagagttttaatcatcactgaggaaaaacatagatatctaatactgactggagtattagtcaaggcttatttcacacacaattttatcagaaaccaaagtagtttaaaacagctctccccttattagtaatgcattggagggtttactttaccatgtaccttgctgagcactgtaccttgttaatctcatttacttgtaatgagaaccacacagcgggtagttttattggttctattttacctacatgacaaaactgaagcataaaaacacttagtaagttttcagtgtcatgcacaactaggaagtgacatggccagaatataagcccagtcaccatcactctataacctgcgcttttaacaacttcagggcatgacacatttggccggtcagtagaacccatgctgtgatttgtttttgcagtggtggtgatgactgccttgttgaatccactttttattctattccattttggggacacaattctgcaagatgattcttcattaggaaacagagatgagttattgaccaacacagaaagaaaaagagtttgttgctccacactgggattaaacctatgatcttggcctaattaacactagctagtaagtgtccaagctgatcatctctacaacatttcaataacagaaaacaacaattttcaaaattagttacttacaattatgtagaaatgcctctaaaacacagtattttccttatattacaaaaacaaaaattataattggttttgtcctcttttgagagtttgcatggtgttactccctgcatagtgaagaaaacattttatttaagtagatggatctaagtttttcatgaacaaaggaatgacatttgaaatcaatcctaccctagtccaggagaatgcattagattaacctagtagaggtcttatttcaccctgagttttctatgatcgtgattctctgctggaggagtaattgtgaaatagatctctctgggaactggcttcctagtccaatcagctcttttaccaatgaacacttccttgtgatatagatgtttatggccgagaggatcctgggtaggaaacacatttgaatggtatttactaagatactaaaatccttggacttcactctaattttagtgccatttagaactcaaggtctcagtaaaagtagaaataaagcctgttaacaaaacacaaactgaatattaaaaatgtaactggattttcaaagaaatgtttactggtattacctgtagatgtatattctttattatgatcttttgtgtaaagtctggcagacaaatgcaatatctaattgttgagtccaatatcacaagcagtacaaaagtataaaaaagacttggccttttctaatgtgttaaaatactttatgctggtaataacactaagagtagggcactagaaattttaagtgaagataatgtgttgcagttactgcactcaatggcttactattataaaccaaaactgggatcactaagctccagtcagtcaaaatgatcaaaattattgaagagaataagcaattctgttctttattaggacacagtagatacagactacaaagtggagtgtgcttaataagaggtagcatttgttaagtgtcaattactctattatcccttggagcttctcaaaataaccatataaggtgtaagatgttaaaggttatggttacactcagtgcacaggtaagctaataggctgagagaagctaaattacttactggggtctcacagtaagaaagtgagctgaagtttcagcccagatttaactggattctgggctctttattcatgttacttcatgaatctgtttctcaattgtgcagaaaaaagggggctatttataagaaaagcaataaacaaacaagtaatgatctcaaataagtaatgcaagaaatagtgagatttcaaaatcagtggcagcgatttctcagttctgtcctaagtggccttgctcaatcacctgctatcttttagtggagctttgaaattatgtttcagacaacttcgattcagttctagaatgtttgactcagcaaattcacaggctcatctttctaacttgatggtgaatatggaaattcagctaaatggatgttaataaaattcaaacgttttaaggacagatggaaatgacagaattttaaggtaaaatatatgaaggaatataagataaaggatttttctaccttcagcaaaaacatacccactaattagtaaaattaataggcgaaaaaaagttgcatgctcttatactgtaatgattatcattttaaaactagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatag

SEQ ID NO: 6 - DNA, 4241 bp, Artificial Sequence
  Description of Artificial Sequence Synthetic polynucleotide
atgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacaggtaagaacagtatgaagaaaagagatgaagcctctgtcttttttacatgttaacagtctcatattagtccttcagaataattctacaatcctaaaataacttagccaacttgctgaattgtattacggcaaggtttatatgaattcatgactgatatttagcaaatgattaattaatatgttaataaaatgtagccaaaacaatatcttaccttaatgcctcaatttgtagatctcggtatttgtgaaataataacgtaaacttcgtttaaaaggattcttcttcctgtctttgagaaagtacggcactgtgcagggggagaggttgattgtgaaaaatcagaggtagatgagaatcttactgagggctgagggttctttaaccttggtggatctcaacattggttgcacattaaaatcacctgctgcaagcccttgacgaatcttacttagaagatgacaacacagaacaattaaatcagaatctctggggagaatagggcaccagtattttttgagctcccaccatgattccaaagtgcagccaaatttgagaaccactgctaaaagctcaagcttcagattgaccagcttttccatctcacctatcgcctaaagaccaaattggataaatgtgttcattacgacagatgggtactatttaaagatgagtaaacacaatatacttaggctcgtcagactgagagttttaatcatcactgaggaaaaacatagatatctaatactgactggagtattagtcaaggcttatttcacacacaattttatcagaaaccaaagtagtttaaaacagctctccccttattagtaatgcattggagggtttactttaccatgtaccttgctgagcactgtaccttgttaatctcatttacttgtaatgagaaccacacagcgggtagttttattggttctattttacctacatgacaaaactgaagcataaaaacacttagtaagttttcagtgtcatgcacaactaggaagtgacatggccagaatataagcccagtcaccatcactctataacctgcgcttttaacaacttcagggcatgacacatttggccggtcagtagaacccatgctgtgatttgtttttgcagtggtggtgatgactgccttgttgaatccactttttattctattccattttggggacacaattctgcaagatgattcttcattaggaaacagagatgagttattgaccaacacagaaagaaaaagagtttgttgctccacactgggattaaacctatgatcttggcctaattaacactagctagtaagtgtccaagctgatcatctctacaacatttcaataacagaaaacaacaattttcaaaattagttacttacaattatgtagaaatgcctctaaaacacagtattttccttatattacaaaaacaaaaattataattggttttgtcctcttttgagagtttgcatggtgttactccctgcatagtgaagaaaacattttatttaagtagatggatctaagtttttcatgaacaaaggaatgacatttgaaatcaatcctaccctagtccaggagaatgcattagattaacctagtagaggtcttatttcaccctgagttttctatgatcgtgattctctgctggaggagtaattgtgaaatagatctctctgggaactggcttcctagtccaatcagctcttttaccaatgaacacttccttgtgatatagatgtttatggccgagaggatccttatgtttcagacaacttcgattcagttctagaatgtttgactcagcaaattcacaggctcatctttctaacttgatggtgaatatggaaattcagctaaatggatgttaataaaattcaaacgttttaaggacagatggaaatgacagaattttaaggtaaaatatatgaaggaatataagataaaggatttttctaccttcagcaaaaacatacccactaattagtaaaattaataggcgaaaaaaagttgcatgctcttatactgtaatgattatcattttaaaactagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatag

SEQ ID NO: 7 - DNA, 5602 bp, Artificial Sequence
  Description of Artificial Sequence Synthetic polynucleotide
atgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacaggtaagaacagtatgaagaaaagagatgaagcctctgtcttttttacatgttaacagtctcatattagtccttcagaataattctacaatcctaaaataacttagccaacttgctgaattgtattacggcaaggtttatatgaattcatgactgatatttagcaaatgattaattaatatgttaataaaatgtagccaaaacaatatcttaccttaatgcctcaatttgtagatctcggtatttgtggatccagtatattaataaaatccctttttgtattcaatgagggaaacacataattttcatcaattagcagcttattggaatatctgcatgatggtttaacacttttaagtgttgactaaagattaattttacagaaaatagaaaaagaaatatgtttctgtctggaggaatgatttattgttgacccctaaattgaaatattttactagtggcttaatggaaagatgatgaaagatgatgaaattaatgtagaagcttaactagaaaatcaggtgacctgatatctacatctgtatccttcattggccacccagcattcattaatgaatcagatgatggaatagatcaagtttcctaggaacacagtgaatattaaaagaaaacaaagggagcctagcacctagaagacctagtttatatttcaaagtatatttggatgtaacccaattttaaacatttcctcacttgtctctcttaaagccttgccaacagcaaggacagagaaccaaaaatagtgtatatatgaataaatgcttattacagaatctgctgactggcacatgctttgtgtgtaatgggttctcataaacacttgttgaatgaacacacataagtgaaagagcatggctaggcttcatcccttggtcaaatatggggtgctaaagaaaagcaggggaaatacattgggacactaacaaaaaaaaacagttaatttaggtaaaagataaaatacaccacagaatgaagaaaagagatgacccagactgctctttaaccttcatgtcctagagaggtttttgatatgaattgcattcagaattgtggaaaggagcccatcttttctcttcattttgattttattaactccaatgggggaattttattcgtgttttggccatatctacttttgatttctacattattctctcttcctttctacctgtatttgtcctaataaattgttgacttattaattcactacttcctcacagcttttttttggctttacaaatccactggaaaggtatatgggtgtatcactttgtgtatttcggtgtgcatgtgtagaggggacaaaaatcctctctcaaactataaatattgagtatttgtgtattgaacatttgctataactactaggtttcttaaataatcttaatatataaaatgatatagaaaaagggaaattatagttcgtattattcatctaagtgaagagattaaaacccagggagtaaataaattgtctaaggactaaggttgtatactatttaggtgatagatatggggcaaccgtatgggttttatgattaacaaataaacttctcaccactctaccatatcaacttttccataaaagagagctatagtattctttgcttaaataaatttgattagtgcatgacttcttgaaaacatataaagcaaaagtcacatttgattctatcagaaaagtgagtaagccatggcccaaacaaaagatgcattaaaatattctggaatgatggagctaaaagtaagaaaaatgactttttaaaaaagtttactgttaggaattgtgaaattatgctgaattttagttgcattataatttttgtcagtcatacggtctgacaacctgtcttatttctatttccccatatgaggaatgctagttaagtatggatattaactattactacttagatgcattgaagttgcataatatggataatacttcactggttccctgaaaatgtttagttagtaataagtctcttacactatttgttttgtccaataatttatattttctgaagacttaactctagaatacactcatgtcaaaatgaaagaatttcattgcaaaatattgcttggtacatgacgcatacctgtatttgttttgtgtcacaacatgaaaaatgatggtttattagaagtttcattgggtaggaaacacatttgaatggtatttactaagatactaaaatccttggacttcactctaattttagtgccatttagaactcaaggtctcagtaaaagtagaaataaagcctgttaacaaaacacaaactgaatattaaaaatgtaactggattttcaaagaaatgtttactggtattacctgtagatgtatattctttattatgatcttttgtgtaaagtctggcagacaaatgcaatatctaattgttgagtccaatatcacaagcagtacaaaagtataaaaaagacttggccttttctaatgtgttaaaatactttatgctggtaataacactaagagtagggcactagaaattttaagtgaagataatgtgttgcagttactgcactcaatggcttactattataaaccaaaactgggatcactaagctccagtcagtcaaaatgatcaaaattattgaagagaataagcaattctgttctttattaggacacagtagatacagactacaaagtggagtgtgcttaataagaggtagcatttgttaagtgtcaattactctattatcccttggagcttctcaaaataaccatataaggtgtaagatgttaaaggttatggttacactcagtgcacaggtaagctaataggctgagagaagctaaattacttactggggtctcacagtaagaaagtgagctgaagtttcagcccagatttaactggattctgggctctttattcatgttacttcatgaatctgtttctcaattgtgcagaaaaaagggggctatttataagaaaagcaataaacaaacaagtaatgatctcaaataagtaatgcaagaaatagtgagatttcaaaatcagtggcagcgatttctcagttctgtcctaagtggccttgctcaatcacctgctatcttttagtggagctttgaaattatgtttcagacaacttcgattcagttctagaatgtttgactcagcaaattcacaggctcatctttctaacttgatggtgaatatggaaattcagctaaatggatgttaataaaattcaaacgttttaaggacagatggaaatgacagaattttaaggtaaaatatatgaaggaatataagataaaggatttttctaccttcagcaaaaacatacccactaattagtaaaattaataggcgaaaaaaagttgcatgctcttatactgtaatgattatcattttaaaactagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatag

SEQ ID NO: 8 - DNA, 4679 bp, Artificial Sequence
  Description of Artificial Sequence Synthetic polynucleotide
atgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacaggtaagaacagtatgaagaaaagagatgaagcctctgtcttttttacatgttaacagtctcatattagtccttcagaataattctacaatcctaaaataacttagccaacttgctgaattgtattacggcaaggtttatatgaattcatgactgatatttagcaaatgattaattaatatgttaataaaatgtagccaaaacaatatcttaccttaatgcctcaatttgtagatctcggtatttgtggatcccttcctttctacctgtatttgtcctaataaattgttgacttattaattcactacttcctcacagcttttttttggctttacaaatccactggaaaggtatatgggtgtatcactttgtgtatttcggtgtgcatgtgtagaggggacaaaaatcctctctcaaactataaatattgagtatttgtgtattgaacatttgctataactactaggtttcttaaataatcttaatatataaaatgatatagaaaaagggaaattatagttcgtattattcatctaagtgaagagattaaaacccagggagtaaataaattgtctaaggactaaggttgtatactatttaggtgatagatatggggcaaccgtatgggttttatgattaacaaataaacttctcaccactctaccatatcaacttttccataaaagagagctatagtattctttgcttaaataaatttgattagtgcatgacttcttgaaaacatataaagcaaaagtcacatttgattctatcagaaaagtgagtaagccatggcccaaacaaaagatgcattaaaatattctggaatgatggagctaaaagtaagaaaaatgactttttaaaaaagtttactgttaggaattgtgaaattatgctgaattttagttgcattataatttttgtcagtcatacggtctgacaacctgtcttatttctatttccccatatgaggaatgctagttaagtatggatattaactattactacttagatgcattgaagttgcataatatggataatacttcactggttccctgaaaatgtttagttagtaataagtctcttacactatttgttttgtccaataatttatattttctgaagacttaactctagaatacactcatgtcaaaatgaaagaatttcattgcaaaatattgcttggtacatgacgcatacctgtatttgttttgtgtcacaacatgaaaaatgatggtttattagaagtttcattgggtaggaaacacatttgaatggtatttactaagatactaaaatccttggacttcactctaattttagtgccatttagaactcaaggtctcagtaaaagtagaaataaagcctgttaacaaaacacaaactgaatattaaaaatgtaactggattttcaaagaaatgtttactggtattacctgtagatgtatattctttattatgatcttttgtgtaaagtctggcagacaaatgcaatatctaattgttgagtccaatatcacaagcagtacaaaagtataaaaaagacttggccttttctaatgtgttaaaatactttatgctggtaataacactaagagtagggcactagaaattttaagtgaagataatgtgttgcagttactgcactcaatggcttactattataaaccaaaactgggatcactaagctccagtcagtcaaaatgatcaaaattattgaagagaataagcaattctgttctttattaggacacagtagatacagactacaaagtggagtgtgcttaataagaggtagcatttgttaagtgtcaattactctattatcccttggagcttctcaaaataaccatataaggtgtaagatgttaaaggttatggttacactcagtgcacaggtaagctaataggctgagagaagctaaattacttactggggtctcacagtaagaaagtgagctgaagtttcagcccagatttaactggattctgggctctttattcatgttacttcatgaatctgtttctcaattgtgcagaaaaaagggggctatttataagaaaagcaataaacaaacaagtaatgatctcaaataagtaatgcaagaaatagtgagatttcaaaatcagtggcagcgatttctcagttctgtcctaagtggccttgctcaatcacctgctatcttttagtggagctttgaaattatgtttcagacaacttcgattcagttctagaatgtttgactcagcaaattcacaggctcatctttctaacttgatggtgaatatggaaattcagctaaatggatgttaataaaattcaaacgttttaaggacagatggaaatgacagaattttaaggtaaaatatatgaaggaatataagataaaggatttttctaccttcagcaaaaacatacccactaattagtaaaattaataggcgaaaaaaagttgcatgctcttatactgtaatgattatcattttaaaactagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatag

SEQ ID NO: 9 - DNA, 3679 bp, Artificial Sequence
  Description of Artificial Sequence Synthetic polynucleotide
atgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacaggtaagaacagtatgaagaaaagagatgaagcctctgtcttttttacatgttaacagtctcatattagtccttcagaataattctacaatcctaaaataacttagccaacttgctgaattgtattacggcaaggtttatatgaattcatgactgatatttagcaaatgattaattaatatgttaataaaatgtagccaaaacaatatcttaccttaatgcctcaatttgtagatctcggtatttgtggatcctgggtaggaaacacatttgaatggtatttactaagatactaaaatccttggacttcactctaattttagtgccatttagaactcaaggtctcagtaaaagtagaaataaagcctgttaacaaaacacaaactgaatattaaaaatgtaactggattttcaaagaaatgtttactggtattacctgtagatgtatattctttattatgatcttttgtgtaaagtctggcagacaaatgcaatatctaattgttgagtccaatatcacaagcagtacaaaagtataaaaaagacttggccttttctaatgtgttaaaatactttatgctggtaataacactaagagtagggcactagaaattttaagtgaagataatgtgttgcagttactgcactcaatggcttactattataaaccaaaactgggatcactaagctccagtcagtcaaaatgatcaaaattattgaagagaataagcaattctgttctttattaggacacagtagatacagactacaaagtggagtgtgcttaataagaggtagcatttgttaagtgtcaattactctattatcccttggagcttctcaaaataaccatataaggtgtaagatgttaaaggttatggttacactcagtgcacaggtaagctaataggctgagagaagctaaattacttactggggtctcacagtaagaaagtgagctgaagtttcagcccagatttaactggattctgggctctttattcatgttacttcatgaatctgtttctcaattgtgcagaaaaaagggggctatttataagaaaagcaataaacaaacaagtaatgatctcaaataagtaatgcaagaaatagtgagatttcaaaatcagtggcagcgatttctcagttctgtcctaagtggccttgctcaatcacctgctatcttttagtggagctttgaaattatgtttcagacaacttcgattcagttctagaatgtttgactcagcaaattcacaggctcatctttctaacttgatggtgaatatggaaattcagctaaatggatgttaataaaattcaaacgttttaaggacagatggaaatgacagaattttaaggtaaaatatatgaaggaatataagataaaggatttttctaccttcagcaaaaacatacccactaattagtaaaattaataggcgaaaaaaagttgcatgctcttatactgtaatgattatcattttaaaactagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatag

SEQ ID NO: 10 - DNA, 2730 bp, Artificial Sequence
  Description of Artificial Sequence Synthetic polynucleotide
atgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacaggtaagaacagtatgaagaaaagagatgaagcctctgtcttttttacatgttaacagtctcatattagtccttcagaataattctacaatcctaaaataacttagccaacttgctgaattgtattacggcaaggtttatatgaattcatgactgatatttagcaaatgattaattaatatgttaataaaatgtagccaaaacaatatcttaccttaatgcctcaatttgtagatctcggtatttgtggatccttatgtttcagacaacttcgattcagttctagaatgtttgactcagcaaattcacaggctcatctttctaacttgatggtgaatatggaaattcagctaaatggatgttaataaaattcaaacgttttaaggacagatggaaatgacagaattttaaggtaaaatatatgaaggaatataagataaaggatttttctaccttcagcaaaaacatacccactaattagtaaaattaataggcgaaaaaaagttgcatgctcttatactgtaatgattatcattttaaaactagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatag

SEQ ID NO: 11 - DNA, 7377 bp, Artificial Sequence
  Description of Artificial Sequence Synthetic polynucleotide
cgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagctcggagattgggctcgcaccgctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgaagcttgctagcaccatgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacaggtaagaacagtatgaagaaaagagatgaagcctctgtcttttttacatgttaacagtctcatattagtccttcagaataattctacaatcctaaaataacttagccaacttgctgaattgtattacggcaaggtttatatgaattcatgactgatatttagcaaatgattaattaatatgttaataaaatgtagccaaaacaatatcttaccttaatgcctcaatttgtagatctcggtatttgtggatcctgggtaggaaacacatttgaatggtatttactaagatactaaaatccttggacttcactctaattttagtgccatttagaactcaaggtctcagtaaaagtagaaataaagcctgttaacaaaacacaaactgaatattaaaaatgtaactggattttcaaagaaatgtttactggtattacctgtagatgtatattctttattatgatcttttgtgtaaagtctggcagacaaatgcaatatctaattgttgagtccaatatcacaagcagtacaaaagtataaaaaagacttggccttttctaatgtgttaaaatactttatgctggtaataacactaagagtagggcactagaaattttaagtgaagataatgtgttgcagttactgcactcaatggcttactattataaaccaaaactgggatcactaagctccagtcagtcaaaatgatcaaaattattgaagagaataagcaattctgttctttattaggacacagtagatacagactacaaagtggagtgtgcttaataagaggtagcatttgttaagtgtcaattactctattatcccttggagcttctcaaaataaccatataaggtgtaagatgttaaaggttatggttacactcagtgcacaggtaagctaataggctgagagaagctaaattacttactggggtctcacagtaagaaagtgagctgaagtttcagcccagatttaactggattctgggctctttattcatgttacttcatgaatctgtttctcaattgtgcagaaaaaagggggctatttataagaaaagcaataaacaaacaagtaatgatctcaaataagtaatgcaagaaatagtgagatttcaaaatcagtggcagcgatttctcagttctgtcctaagtggccttgctcaatcacctgctatcttttagtggagctttgaaattatgtttcagacaacttcgattcagttctagaatgtttgactcagcaaattcacaggctcatctttctaacttgatggtgaatatggaaattcagctaaatggatgttaataaaattcaaacgttttaaggacagatggaaatgacagaattttaaggtaaaatatatgaaggaatataagataaaggatttttctaccttcagcaaaaacatacccactaattagtaaaattaataggcgaaaaaaagttgcatgctcttatactgtaatgattatcattttaaaactagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatagcggccgctctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagtcgaaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatg

(19) United States
(12) Patent Application Publication
(10) Pub. No.: US 2019/0111154 A1
(43) Pub. Date: Apr. 18, 2019

(54) TREATMENT OF NEUROPATHY WITH DNA CONSTRUCT EXPRESSING HGF ISOFORMS WITH REDUCED INTERFERENCE FROM GABAPENTINOIDS

(71) Applicant: ViroMed Co., Ltd., Seoul (KR)

(72) Inventors: Sunyoung KIM, Pocheon-si (KR); Junghun LEE, Seoul (KR)

(21) Appl. No.: 16/161,101

(22) Filed: Oct. 16, 2018

Related U.S. Application Data
(60) Provisional application No. 62/574,100, filed on Oct. 18, 2017.

(51) Int. Cl. A61K 48/00; C12N 15/62; A61P 29/02; A61K 9/00; A61P 25/04; A61P 25/02

(52) U.S. Cl. CPC A61K 48/0016; C12N 15/62; A61P 29/02; A61K 9/0019; A61K 48/0075; A61K 48/0083; A61K 48/0091; A61P 25/02; A61P 25/04

(57) ABSTRACT

The present invention relates to methods of treating neuropathy patients who have been administered a gabapentinoid. In particular, the methods involve administering a nucleic acid construct encoding human HGF proteins after discontinuing gabapentinoid. The present invention provides a novel method for a specific patient population to achieve a better therapeutic outcome by avoiding interference of therapeutic effects by gabapentinoids.

### 1. CROSS-REFERENCE TO RELATED APPLICATIONS

This application claims the benefit of U.S. Provisional Application No. 62/574,100, filed Oct. 18, 2017, which is incorporated by reference in its entirety.

### 2. SEQUENCE LISTING

The instant application contains a Sequence Listing which has been submitted via EFS-Web and is hereby incorporated by reference in its entirety. Said ASCII copy, created on Sep. 28, 2018, is named 37236US_CRF_sequencelisting, and is 75,361 bytes bytes in size.

### 3. BACKGROUND

Gabapentinoids are a class of drugs that are derivatives of the inhibitory neurotransmitter GABA (γ-aminobutyric acid). Several gabapentinoids have been developed and clinically-approved, including gabapentin (Neurontin) and pregabalin (Lyrica) as well as a gabapentin prodrug, gabapentin enacarbil (Horizant).

Gabapentinoids are believed to act mainly on the α2δ subunit of pre-synaptic calcium channels and inhibit neuronal calcium influx. This results in a reduction in the release of excitatory neurotransmitters such as glutamate, substance P, and calcitonin gene-related peptide from nerve fibers, thus suppressing neuronal excitability after nerve or tissue injury. These drugs have been used for the treatment of a variety of conditions associated with nerve damage, such as neuropathic pain, as well as various other nervous system disorders including epilepsy, fibromyalgia, generalized anxiety disorder, and restless leg syndrome. They have also been suggested to be effective in treatment of migraine, social phobia, panic disorder, mania, bipolar disorder, and alcohol withdrawal.

Recently, it was demonstrated that neuropathic pain can be treated with a DNA construct that expresses two isoforms of human HGF protein (i.e., pCK-HGF-X7, also called “VM202”). In a phase II clinical trial, injections of VM202 into the calf muscle of patients with diabetic peripheral neuropathy were shown to significantly reduce pain—two days of treatment, spaced two weeks apart, were sufficient to provide symptomatic relief with improvement in quality of life for 3 months. Kessler et al., Annals Clin. Transl. Neurology 2(5):465-478 (2015).

Although VM202 was demonstrated to be effective in treating patients with diabetic peripheral neuropathy, further analysis of the phase II clinical trial data demonstrated that VM202 was more effective in relieving pain in patients not taking pregabalin or gabapentin than in patients who were taking a gabapentinoid. Kessler et al., Annals Clin. Transl. Neurology 2(5):465-478 (2015). However, the post hoc analysis could not elucidate the physiological mechanism underlying this observation. In particular, the data could not predict whether prior administration of a gabapentinoid would preclude later efficacy of VM202, nor predict how to administer VM202 efficaciously to patients who had previously taken gabapentinoids.

There is, therefore, a need for methods of administering VM202 efficaciously to a patient who has previously been administered a gabapentinoid.

### 4. SUMMARY

The present invention is based on a novel finding related to interference of therapeutic effects of the nucleic acid construct encoding HGF (e.g., VM202) on neuropathy by gabapentinoids. Specifically, we have discovered that gabapentinoids have deleterious effects on VM202 in an animal model of neuropathic pain when gabapentinoid is administered at the time of and shortly after administration of VM202. This inhibitory effect of gabapentinoid administered together and shortly after VM202 lasted even after discontinuation of gabapentinoid. However, gabapentinoid administered after more than one week after VM202 did not affect therapeutic efficacy of VM202. These results suggest that it is important to discontinue gabapetinoid treatment before, during and for a few days after administration of VM202 to maximize the therapeutic efficacy and potency of VM202.

Accordingly, in a first aspect, methods are presented for treating neuropathy with a nucleic acid construct encoding isoforms of HGF (e.g., VM202) in patients who have been administering gabapentinoids. Specifically, the methods involve discontinuing administration of gabapentinoids for certain periods before, during, and after administration of the nucleic acid construct. Thus, the present invention provides a novel method for a specific patient population to achieve a better therapeutic outcome by avoiding interference by gabapentinoids.

Specifically, some embodiments of the present invention are directed to a method of treating neuropathy, comprising: (1) selecting a patient with neuropathy who has been administered a gabapentinoid, (2) discontinuing gabapentinoid administration to the patient, and (3) administering VM202 to the patient. In some embodiments, the method further comprises the step of withholding gabapentinoid administration for at least a week after the step of administering VM202. In some embodiments, the method further comprises the step of withholding gabapentinoid administration for at least 10 days after the step of administering VM202.

In some embodiments, the step of discontinuing gabapentinoid administration comprises tapering gabapentinoid administration. In some cases, the step of administering VM202 is performed after a complete cessation of gabapentinoid administration. In some cases, the step of administering VM202 is performed at least 1, 2, 3, 4, 5, 7, 14, 21, 30, 60, or 90 days after a complete cessation of gabapentinoid administration.

In some embodiments, the neuropathy is diabetic peripheral neuropathy. In some embodiments, the neuropathy is post-herpetic neuropathy.

In some embodiments, the gabapentinoid is gabapentin or pregabalin.

In some embodiments, the step of administering VM202 comprises administering 8 mg of VM202 per affected limb of the patient, equally divided into a plurality of intramuscular injections and plurality of visits, wherein each of the plurality of intramuscular injections in any single visit is performed at a separate injection site.

In some embodiments, VM202 is administered at a dose of 16 mg equally divided into 64 intramuscular injections, wherein 16 intramuscular injections are administered to separate injection sites on a first calf on a first visit, wherein 16 intramuscular injections are administered to separate injection sites on a second calf on the first visit, wherein 16 intramuscular injections are administered to separate injection sites on the first calf on a second visit, wherein 16 intramuscular injections are administered to separate injection sites on the second calf on the second visit, and wherein each of the 64 intramuscular injections is performed with 0.25 mg of VM202 in a volume of 0.5 ml.

In another aspect, the present invention provides a method of treating neurlpathy by administering VM202, the improvement comprising: selecting a patient with neuropathy who has been administered a gabapentinoid; discontinuing gabapentinoid administration to the patient; and then administering VM202 to the patient.

In yet another aspect, the present invention provides a method of treating neuropathy, comprising the steps of: determining whether a patient with neuropathy has been administered a gabapentinoid within the preceding week; if the patient has been administered a gabapentinoid within the preceding week, discontinuing gabapentinoid administration to the patient, and thereafter administering VM202 to the patient; and if the patient has not been administered a gabapentinoid within the preceding week, administering VM202 to the patient.

Also provided herein is a nucleic acid construct encoding isoforms of HGF (e.g., VM202) for use in a method of treating neuropathy in a patient who has been administered a gabapentinoid, wherein the method comprising discontinuing administration of the gabapentinoid to the patient, and administering the nucleic acid construct to the patient. Some embodiments provide a nucleic acid construct encoding isoforms of HGF (e.g., VM202) for use in a method of treating neuropathy, the method comprising the steps of selecting a patient with neuropathy who has been administered a gabapentinoid, discontinuing the gabapentinoid to the patient, and administering the nucleic acid construct to the patient. Some embodiments provide a nucleic acid construct encoding isoforms of HGF (e.g., VM202) for use in a method of treating neuropathy, the method comprising the steps of selecting a patient with neuropaty who has been administered a gabapentinoid, administering the nucleic acid construct only after discontinuation of the gabapentinoid, and administering no further dose of the gabapentinoid for at least one week.

Also provided herein is the use of a nucleic acid construct encoding isoforms of HGF (e.g., VM202) for the preparation of a medicament for the treatment of neuropathy in a patient who has been administered a gabapentinoid. Some embodiments relate to the use of the nucleic acid construct for the preparation of a medicament for the treatment of neuropathy in a patient who has been administered a gabapentinoid; but discontinued, is discontinuing or will discontinue gabapentinoid administration. Some embodiments related to the use of the nucleic acid construct for the preparation of a medicament for the treatment of neuropathy in a patient who has been administered a gabapentinoid but will discontinue gabapentinoid administration before and for at least one week after administration of the nucleic acid construct.

### 5. BRIEF DESCRIPTION OF THE DRAWINGS

FIG. 1A, reproduced from Kessler et al., Annals Clin. Transl. Neurology 2(5):465-478 (2015), shows time-course change in pain levels measured in all patients in the phase 2 clinical trial at 3, 6, and 9 months after the administration of a high dose of VM202 (8 mg per leg on day 0, 8 mg per leg on day 14; total dose across both legs and both visits, 32 mg), a low dose of VM202 (4 mg per leg on day 0, 4 mg per leg on day 14; total dose across both legs and both visits, 16 mg), or saline (placebo). FIG. 1B, also reproduced from Kessler et al., Annals Clin. Transl. Neurology 2(5):465-478 (2015), shows time-course change in pain levels measured in a group of patients who were not on Lyrica (pregabalin) and/or Neurontin (gabapentin), 3, 6, and 9 months after administering the high dose of VM202, the low dose of VM202, or saline (placebo). Patients who were not on Lyrica and/or Neurontin (FIG. 1B) generally experienced a larger reduction in pain from baselines than the total patient group (FIG. 1A) after administration of the low dose of VM202.

FIG. 2 illustrates the experimental procedure for testing effects of gabapentin on VM202-mediated pain reduction using chronic constriction injury (CCI) mice. Surgical procedure on sciatic nerve (CCI or sham) was performed and a plasmid (pCK or VM202) was administered to 5-week-old male mice on day 0, gabapentin or PBS was injected daily to the mice from day 1 to day 15, and their pain levels were measured by von Frey filament test starting on day 14 through 16.

FIG. 3 provides pain levels (paw withdrawal frequency % on y-axis) measured in four different animal groups. (a) Sham animals without chronic constriction exhibited low levels of pain throughout the time course (“Sham,” line with diamonds); (b) CCI-animals injected with pCK without daily injection of gabapentin had consistently high levels of pain (“pCK-PBS,” line with triangles); (c) CCI-animals injected with pCK with daily injection of gabapentin had temporary reduction in pain levels immediately after gabapentin administration (“pCK+Gabapentin,” line with circles); and (d) CCI-animals injected with VM202 without daily injection of gabapentin had low levels of pain (“VM202,” line with x).

FIG. 4 provides pain levels (paw withdrawal frequency % on y-axis) measured in four different animal groups. (a) Sham animals without chronic constriction exhibited low levels of pain throughout the time course (“Sham,” line with diamonds); (b) CCI-animals injected with pCK without daily injection of gabapentin had high levels of pain (“pCK-PBS,” line with triangles); (c) CCI-animals injected with VM202 without daily injection of gabapentin had low levels of pain (“VM202,” line with x), and (d) CCI-animals injected with VM202 with daily gabapentin administration had temporary decrease in pain immediately after gabapentin administration followed by sharp increase and then gradual decrease in pain levels.

FIG. 5 illustrates the experimental procedure for testing gabapentin effects on VM202-mediated nerve regeneration in a nerve crush mouse model. Nerve crush was induced and VM202 administered to 9-week old male C57BL/6 mice on day 1, gabapentin was injected daily from day 2 to day 6, and nerve pinch test was conducted on day 7.

FIG. 6 shows nerve regeneration (i.e., the length of regenerated nerves (mm)) measured in the nerve crush mouse model. The bars represent extent of nerve regeneration in mice administered, from left to right, (i) negative control for VM202 (pCK vector) and negative control for gabapentin (daily injections with PBS); (ii) VM202 and daily PBS; (iii) pCK and daily injections of gabapentin; and (iv) VM202 and daily injections with gabapentin. Mice treated with VM202 had significantly better nerve regeneration whether treated with PBS or Gabapentin. However, VM202-mediated nerve regeneration was significantly better in the control mice treated with PBS than in the mice treated with gabapentin.

FIG. 7A shows a result from western blot assay of protein samples obtained from Sham mice with daily injection of PBS or gabapentin (lanes 1 and 4); nerve crush mice injected with pCK with additional daily injection of PBS or gabapentin (lanes 2 and 5); and nerve crush mice treated with VM202 with additional daily injection of PBS or gabapentin (lanes 3 and 6). Expressions of c-Jun (top) and GAPDH (bottom) were detected by antibodies specific to each protein. FIG. 7B provides relative levels of c-Jun expression in each sample, calculated by measuring band intensity of c-Jun and comparing the intensity with the band intensity of GAPDH.

FIG. 8A illustrates the experimental procedure for testing VM202-mediated pain reduction in CCI mice when additionally treated with gabapentin during the first two weeks or during the second two weeks (weeks 3-4) after VM202 administration. FIG. 8B provides pain levels (paw withdrawal frequency, # of response) in four different animal groups. (a) Sham animals without chronic constriction exhibited low levels of pain throughout the time course (“Sham”), (b) CCI-animals injected with pCK without daily injection of gabapentin had high levels of pain (“CCI-pCK”), (c) CCI-animals injected with VM202 without daily injection of gabapentin had low levels of pain (“VM202”), (d) CCI-animals injected with VM202 with daily gabapentin administration during the first two weeks had high levels of pain (“CCI-VM202-Gaba1”), and (e) CCI-animals injected with VM202 with daily gabapentin administration during the second two weeks had low levels of pain (“CCI-VM202-Gaba2”).

FIG. 9A illustrates the experimental procedure for testing VM202-mediated pain reduction in CCI mice that are additionally treated with daily injections of gabapentin starting from day 0 (“GP1”), day 3 (“GP2”), day 7 (“GP3”), or day 10 (“GP4”) after VM202 administration. FIG. 9B provides pain levels (paw withdrawal frequency % on y-axis) measured in six different animal groups. (a) CCI-animals injected without VM202 and daily injection of gabapentin had high levels of pain, (b) CCI-animals injected with VM202 without daily injection of gabapentin had low levels of pain, (c) CCI-animals injected with VM202 with daily injection of gabapentin from day 0 had high levels of pain (“GP1”), (d) CCI-animals injected with VM202 with daily injection of gabapentin from day 3 had high levels of pain (“GP2”), (e) CCI-animals injected with VM202 with daily injection of gabapentin from day 7 had high levels of pain (“GP3”), and (f) CCI-animals injected with VM202 with daily injection of gabapentin from day 10 had low levels of pain (“GP4”).

All values are presented as mean±standard error mean (SEM) from three independent experiments. Differences between values were determined by one-way ANOVA followed by Tukey's post-hoc test.

The figures depict various embodiments of the present invention for purposes of illustration only. One skilled in the art will readily recognize from the following discussion that alternative embodiments of the structures and methods illustrated herein may be employed without departing from the principles of the invention described herein.

### 6. DETAILED DESCRIPTION

### 6.1. Definitions

Unless defined otherwise, all technical and scientific terms used herein have the meaning commonly understood by a person skilled in the art to which this invention belongs. As used herein, the following terms have the meanings ascribed to them below.

The term “gabapentinoid(s)” as used herein refers to a class of drugs that are derivatives of the inhibitory neurotransmitter γ-aminobutyric acid (GABA) and which block α2δ subunit-containing voltage-dependent calcium channels. Gabapentinoids include, but are not limited to, clinically approved gabapentinoids, such as gabapentin (Neurontin) and pregabalin (Lyrica) as well as a gabapentin prodrug, gabapentin enacarbil (Horizant).

The term “discontinue” as used herein refers to a process of breaking continuation of drug administration. It includes, but not limited to, abrupt termination of administration and termination of administration by decreasing administration amount and/or frequency over certain periods. Sometimes, the process can involve a temporary increase, decrease or maintenance of amount and/or frequency of administration. The process can also involve switching from one gabapentinoid to another gabapentinoid.

The term “taper” as used herein refers to a method of discontinuing drug administration by gradually reducing the amount or frequency of drug administration toward the end.

The term “isoforms of HGF” as used herein refers to a polypeptide having an amino acid sequence that is at least 80% identical to the amino acid sequence of a naturally occurring HGF polypeptide in an animal. The term includes polypeptides having an amino acid sequence that is at least 80% identical to any full length wild type HGF polypeptide, and includes polypeptides having an amino acid sequence that is at least 80% identical to a naturally occurring HGF allelic variant, splice variant, or deletion variant. Isoforms of HGF preferred for use in the present invention include two or more isoforms selected from the group consisting of full-length HGF (flHGF) (synonymously, fHGF), deleted variant HGF (dHGF), NK1, NK2, and NK4. According to a more preferred embodiment of the present invention, the isoforms of HGF used in the methods described herein include flHGF and dHGF.

The terms “human flHGF”, “flHGF” and “fHGF” are used interchangeably herein to refer to a protein consisting of amino acids 1-728 of the human HGF protein. The sequence of flHGF is provided in SEQ ID NO: 1.

The terms “human dHGF” and “dHGF” are used interchangeably herein to refer to a deleted variant of the HGF protein produced by alternative splicing of the human HGF gene. Specifically, “human dHGF” or “dHGF” refers to a human HGF protein with deletion of five amino acids (F, L, P, S, and S) in the first kringle domain of the alpha chain from the full length HGF sequence. Human dHGF is 723 amino acids in length. The amino acid sequence of human dHGF is provided in SEQ ID NO: 2.

The term “VM202” as used herein refers to a plasmid DNA also called as pCK-HGF-X7 (SEQ ID NO: 11), HGF-X7 cloned into the pCK vector. VM202 was deposited under the terms of the Budapest Treaty at the Korean Culture Center of Microorganisms (KCCM) under accession number KCCM-10361 on Mar. 12, 2002.

The term “vector” as used herein refers to any vehicle for the cloning of and/or transfer of a nucleic acid into a host cell. Vectors include, inter alia, plasmids, viral vectors, lipoplexes (cationic liposome-DNA complex), polyplexes (cationic polymer-DNA complex), and protein-DNA complexes.

The term “expression vector” as used herein refers to a vector designed to enable the expression of an inserted nucleic acid sequence following transformation into the host.

The term “reconstituted” or “reconstitution” refers to the restoration to the original form, e.g., by rehydration, of a substance previously altered for preservation and storage, e.g., the restoration to a liquid state of a DNA plasmid formulation that has been previously dried and stored. The lyophilized composition of the present invention may be reconstituted in any aqueous solution which produces a stable, mono-dispersed solution suitable for administration. Such aqueous solutions include, but are not limited to: sterile water, TE, PBS, Tris buffer or normal saline.

The concentration of reconstituted lyophilized DNA in the methods of the current invention is adjusted depending on many factors, including the amount of a formulation to be delivered, the age and weight of the subject, the delivery method and route and the immunogenicity of the antigen being delivered.

The term “treatment” as used herein refers to all the acts of (a) suppressing neuropathic pain; (b) alleviation of neuropathic pain; and (c) removal of neuropathic pain. In some embodiments, the composition of the present invention can treat neuropathic pain through the growth of neuronal cells or the suppression of neuronal cell death.

The term “therapeutically effective dose” or “effective amount” as used herein refers to a dose or amount that produces the desired effect for which it is administered. In the context of the present methods, a therapeutically effective amount is an amount effective to treat a symptom of neuropathy. The exact dose or amount will depend on the purpose of the treatment, and will be ascertainable by one skilled in the art using known techniques (see, e.g., Lloyd (1999) The Art, Science and Technology of Pharmaceutical Compounding).

The term “sufficient amount” as used herein refers to an amount sufficient to produce a desired effect.

The term “degenerate sequence” as used herein refers to a nucleic acid sequence that can be translated to provide an amino acid sequence identical to that translated from the reference nucleic acid sequence.

### 6.2. Other Interpretational Conventions

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless otherwise indicated, reference to a compound that has one or more stereocenters intends each stereoisomer, and all combinations of stereoisomers, thereof.

### 6.3. Methods of Treating Neuropathy in Patients Administered Gabapentinoids

In a first aspect, methods are presented for treating neuropathy in patients who have been administered a gabapentinoid. The methods comprise selecting a patient with neuropathy who has been administered a gabapentinoid, discontinuing gabapentinoid administration, and administering a therapeutically effective amount of a nucleic acid construct that expresses two isoforms of a human HGF protein. In preferred embodiments, the nucleic acid construct is VM202.

6.3.1. Patients with Neuropathy

In the methods described herein, the patients selected for treatment have neuropathy. The patients can have peripheral neuropathy, cranial neuropathy, autonomic neuropathy or focal neuropathy. The neuropathy can be caused by diseases, injuries, infections or vitamin deficiency states. For example, the neuropathy can be caused by diabetes, vitamin deficiencies, autoimmune diseases, genetic or inherited disorders, amyloidosis, uremia, toxins or poisons, trauma or injury, tumors, or can be idiopathic.

In currently preferred embodiments, the patients have diabetic peripheral neuropathy.

6.3.2. Patients Who have been Administered a Gabapentinoid

Patients who have been administered gabapentinoids can be selected by various methods known in the art. For example, the selection can be made based on information obtained from the patient or a guardian of the patient as a part of the response to standardized questionnaires or during interview. The selection can be also based on information obtained from medical, clinical, prescription or insurance records associated with the patient, or any other record, or from a medical professional for the patient. Information relevant for the selection can include, but is not limited to, the name of an administered drug, and its dosage, frequency, route of administration, date of first administration, date of last administration, etc. Alternatively, the selection can be based on information obtained by diagnosis, such as a blood test.

In some embodiments, the patient's latest exposure to gabapentinoids will have been less than three months before the time of the selection. In some embodiments, the patient's latest exposure to gabapentinoids is less than 1, 2, 3, 4, 5, 6, 7, or 8 weeks before the time of the selection. In some embodiments, the patient's latest exposure to gabapentinoids is less than 1, 5, 10, 15, 20, 25, 30, 35, 40, 45, or 50 days before the time of selection.

In some embodiments, the patient's latest exposure to gabapentinoids is less than three months before the time of VM202 first administration. In some embodiments, the patient's latest exposure to gabapentinoids is less than 1, 2, 3, 4, 5, 6, 7, or 8 weeks before the time of VM202 first administration. In some embodiments, the patient's latest exposure to gabapentinoids is less than 1, 5, 10, 15, 20, 25, 30, 35, 40, 45, or 50 days before the time of VM202 first administration.

In some embodiments, the patient's last exposure to more than 30%, 40%, 50%, 60%, 70%, 80%, 90% or 95% of the prescribed dose of gabapentinoids is less than three months before the time of the selection. In some embodiments, the patient's last exposure to more than 30%, 40%, 50%, 60%, 70%, 80%, 90% or 95% of the prescribed dose of gabapentinoids is less than 1, 2, 3, 4, 5, 6, 7, or 8 weeks before the time of the selection. In some cases, the patient's last exposure to more than 30%, 40%, 50%, 60%, 70%, 80%, 90% or 95% of the prescribed dose of gabapentinoids is less than 1, 5, 10, 15, 20, 25, 30, 35, 40, 45, or 50 days before the time of the selection.

In some embodiments, the patient's last exposure to more than 30%, 40%, 50%, 60%, 70%, 80%, 90% or 95% of the prescribed dose of gabapentinoids is less than three months before the time of VM202 first administration. In some embodiments, the patient's last exposure to more than 30%, 40%, 50%, 60%, 70%, 80%, 90% or 95% of the prescribed dose of gabapentinoids is less than 1, 2, 3, 4, 5, 6, 7, or 8 weeks before the time of VM202 first administration. In some cases, the patient's last exposure to more than 30%, 40%, 50%, 60%, 70%, 80%, 90% or 95% of the prescribed dose of gabapentinoids is less than 1, 5, 10, 15, 20, 25, 30, 35, 40, 45, or 50 days before the time of VM202 first administration.

In some embodiments, patients previously exposed to gabapentinoids for more than 3, 5, 7, 14, 21, 28, or 35 days are selected to be patients who have been administering gabapentinoids. In some embodiments, patients previously exposed to gabapentinoids for more than 2, 3, 4, 5, 6, 12, or 18 months are selected to be patients who have been administering gabapentinoids.

In some embodiments, patients previously exposed to more than 30%, 40%, 50%, 60%, 70%, 80%, 90% or 95% of the prescribed dose of gabapentinoids for more than 3, 5, 7, 14, 21, 28, or 35 days are selected to be patients who have been administering gabapentinoids. In some embodiments, patients previously exposed to more than 30%, 40%, 50%, 60%, 70%, 80%, 90% or 95% of the prescribed dose of gabapentinoids for more than 2, 3, 4, 5, 6, 12, or 18 months are selected to be patients who have been administering gabapentinoids.

6.3.3. Discontinuation of Gabapentinoids

Once a patient is selected, gabapentinoid administration is discontinued. In some embodiments, gabapentinoid administration is discontinued by completely ceasing gabapentinoid administration.

In some embodiments, gabapentinoid administration is discontinued by tapering gabapentinoid administration. In some embodiments, the dose of gabapentinoid is reduced over 1, 2, 3, 4, 5, 6, 7, or 8 weeks. In some embodiments, the dose of gabapentinoid is reduced over 1, 2, 3, 4, or 5 months. In some embodiments, the frequency of gabapentinoid administration is reduced over 1, 2, 3, 4, 5, 6, 7, or 8 weeks. In some embodiments, the frequency of gabapentinoid administration is reduced over 1, 2, 3, 4, or 5 months.

In various embodiments, gabapentinoid administration is reduced 10-100 mg per week, 20-100 mg per week, 20-90 mg per week, 30-80 mg per week, 40-80 mg per week, 50-75 mg per week, 55-70 mg per week, 55-65 mg per week, or about 60 mg per week.

In some embodiments, gabapentinoid administration is reduced at a rate of less than 500 mg every four days, 450 mg every four days, 400 mg every four days, 350 mg every four days, 300 mg every four days, 250 mg every four days, 200 mg every four days, 150 mg every four days, 100 mg every four days, or 50 mg every four days.

In some embodiments, gabapentinoid administration is reduced at a rate of less than 500 mg every three days, 450 mg every three days, 400 mg every three days, 350 mg every three days, 300 mg every three days, 250 mg every three days, 200 mg every three days, 150 mg every three days, 100 mg every three days, or 50 mg every three days.

In some embodiments, gabapentinoid administration is reduced at a rate of less than 500 mg every two days, 450 mg every two days, 400 mg every two days, 350 mg every two days, 300 mg every two days, 250 mg every two days, 200 mg every two days, 150 mg every two days, 100 mg every two days, 50 mg every two days, 25 mg every two days, 10 mg every two days, or 5 mg every two days.

In some embodiments, the gabapentinoid administration is reduced at a rate of less than 500 mg every day, 450 mg every day, 400 mg every day, 350 mg every day, 300 mg every day, 250 mg per day, 200 mg per day, 150 mg per day, 100 mg per day, 50 mg per day, 25 mg per day, 10 mg per day, 5 mg per day, or 2 mg per day.

In some embodiments, the rate of reducing gabapentinoid administration is adjusted based on patient's response to the reduction. For example, specific rate can be determined based on withdrawal symptoms of the patient, such as rebound anxiety, insomnia, headache, nervousness, depression, pain, increased sweating, dizziness, etc. In some embodiments, specific rate can be determined based on symptoms associated with neuropathy, such as pain.

In some cases, the amount of gabapentinoid administration can be temporarily increased or maintained at the same level during the discontinuation based on patient's response. For example, the amount of gabapentinoid administration can be temporarily increased or held at the same level based on patient's withdrawal symptoms, such as rebound anxiety, insomnia, headache, nervousness, depression, pain, increased sweating, dizziness, etc. In some cases, the amount of gabapentinoid administration can be temporarily increased or held at the same level based on patient's symptoms associated with neuropathy, such as pain.

In some embodiments, the rate of reducing gabapentinoid administration can be determined based on the patient's past exposure to gabapentinoids. For example, specific rate can be determined based on dose or frequency of gabapentinoid administration, or amount or length of previous exposure to gabapentinoids.

6.3.4. Administration of Nucleic Acid Construct Encoding Two Hepatocyte Growth Factor (HGF) Isoforms

The selected patient is administered a therapeutically effective amount of a nucleic acid construct that expresses two isoforms of a human HGF protein.

The patient can be administered the nucleic acid construct after discontinuing gabapentinoid administration to the patient.

In various embodiments, the nucleic acid construct is administered after a complete cessation of gabapentinoid administration. In some embodiments, the nucleic acid construct is administered at least 1, 2, 3, 4, 5, 7, 14, 21, 30, 60, or 90 days after a complete cessation of gabapentinoid administration. In some embodiments, the nucleic acid construct is administered at least 1, 2, 3, 4, 5, 6, 7, or 8 weeks after a complete cessation of gabapentinoid administration. In some embodiments, the nucleic acid construct is administered at least 1, 2, 3, 4, 5, or 6 months after a complete cessation of gabapentinoid administration.

In certain embodiments, the nucleic acid construct is first administered after a complete cessation of gabapentinoid administration. In some embodiments, the first administration of the nucleic acid construct is at least 1, 2, 3, 4, 5, 7, 14, 21, 30, 60, or 90 days after a complete cessation of gabapentinoid administration. In some embodiments, the first administration of the nucleic acid construct is at least 1, 2, 3, 4, 5, 6, 7, or 8 weeks after a complete cessation of gabapentinoid administration. In some embodiments, the first administration of the nucleic acid construct is at least 1, 2, 3, 4, 5, or 6 months after a complete cessation of gabapentinoid administration.

In some embodiments, the nucleic acid construct is first administered while tapering gabapentinoid administration. In some embodiments, the first dose of nucleic acid construct is administered at day 0, 1, 2, 3, 4, 5, 6, 7, 10, 14, 21, 28, or 35 of the tapering regimen. In certain embodiments, the first dose of nucleic acid construct is administered at week 1, 2, 3, 4, 5, or 6 weeks of the tapering process. In some embodiments, the first dose of nucleic acid construct is administered 0, 1, 2, 3, 4, 5, 6, 7, 10, 14, 21, 28, or 35 days after a complete cessation of gabapentinoid administration.

In some embodiments, following administration of the nucleic acid construct expressing two hepatocyte growth factor (HGF) isoforms, gabapentinoid is not again administered for at least 4, 5, 6, 7, 8, 9, 10, 11, 12, 13, or 14 days. In some embodiments, following administration of the nucleic acid construct expressing two hepatocyte growth factor (HGF) isoforms, gabapentinoid is not again administered for at least 1, 2, 3, 4, or 5 weeks. In some embodiments, following administration of the nucleic acid construct expressing two hepatocyte growth factor (HGF) isoforms, gabapentinoid is not again administered for 5, 6, 7, 8, 9, 10, 11, 12, 13, or 14 days. In some embodiments, following administration of the nucleic acid construct expressing two hepatocyte growth factor (HGF) isoforms, gabapentinoid is not again administered for at least 1, 2, 3, 6, 9, 12, 24 or 36 months.

In some embodiments, the nucleic acid construct expressing two hepatocyte growth factor (HGF) isoforms is administered over multiple visits. In such cases, administration of gabapentinoid can be discontinued before each visit. In some embodiments, gabapentinoid is not administered at least for 1, 2, 3, 4, 5, or 6 weeks before each visit. In some embodiments, gabapentinoid is not administered at least for 5, 6, 7, 8, 9, 10 or 15 days before each visit. In some embodiments, gabapentinoid is not administered at least for 1, 2, 3, 4, 5, or 6 weeks after each visit. In some embodiments, gabapentinoid is not administered at least for 5, 6, 7, 8, 9, 10 or 15 days after each visit. In some cases, gabapentinoid is not administered until completion of the nucleic acid construct administration over multiple visits.

6.3.5. Nucleic Acid Construct Expressing Two Hepatocyte Growth Factor (HGF) Isoforms

In the methods described herein, the nucleic acid construct expresses at least two isoforms of a human HGF protein. In some embodiments, the nucleic acid construct expresses two isoforms. In typical embodiments, the nucleic acid construct expresses at least one of flHGF and dHGF. In particular embodiments, the nucleic acid construct expresses both flHGF and dHGF.

6.3.5.1. Expressed Sequences

In some embodiments, the construct expresses two or more isoforms of HGF by comprising an expression regulatory sequence for each isoform coding sequence (CDS). In some embodiments, the construct comprises an internal ribosomal entry site (IRES) between two coding sequences, for example, in the order of (1) expression regulatory sequence—(2) coding sequence of first isomer—(3) IRES—(4) coding sequence of second isomer—(5) transcription termination sequence. IRES allows translation to start at the IRES sequence, thereby allowing expression of two genes of interest from a single construct. In yet further embodiments, a plurality of constructs, each encoding a single isoform of HGF, are used together to induce expression of more than one isoforms of HGF in the subject to whom administered.

Preferred embodiments of the methods use a construct that simultaneously expresses two or more different types of isoforms of HGF—i.e., flHGF and dHGF—by comprising an alternative splicing site. It was previously demonstrated in U.S. Pat. No. 7,812,146, incorporated by reference herein, that a construct encoding two isoforms of HGF (flHGF and dHGF) through alternative splicing has much higher (almost 250 fold higher) expression efficiency than a construct encoding one isoform of HGF (either flHGF or dHGF). In typical embodiments, the construct comprises (i) a first sequence comprising exons 1-4 of a human HGF gene or a degenerate sequence of the first sequence; (ii) a second sequence comprising intron 4 of the human HGF gene or a fragment of the second sequence; and (iii) a third sequence comprising exons 5-18 of the human HGF gene or a degenerate sequence of the third sequence. From the construct, two isoforms of HGF (flHGF and dHGF) can be generated by alternative splicing between exon 4 and exon 5.

In some embodiments, the construct comprises a full sequence of intron 4. In some embodiments, the construct comprises a fragment of intron 4. In preferred embodiments, the construct comprises a nucleotide sequence selected from the group consisting of SEQ ID NO: 3 to SEQ ID NO: 10. The nucleotide sequence of SEQ ID NO: 3 corresponds to a 7113 bp polynucleotide encoding flHGF and dHGF, and including the full sequence of intron 4. The nucleotide sequences of SEQ ID NOS: 4-10 correspond to polynucleotides encoding flHGF and dHGF and including various fragments of intron 4.

Various nucleic acid constructs comprising cDNA corresponding exon 1-18 of human HGF and intron 4 of a human HGF gene or its fragment are named “HGF-X” followed by a unique number as described in U.S. Pat. No. 7,812,146. The HGF-X tested by Applicant includes, but not limited to, HGF-X1, HGF-X2, HGF-X3, HGF-X4, HGF-X5, HGF-X6, HGF-X7, and HGF-X8 having nucleotide sequences of SEQ ID NO: 3 to SEQ ID NO: 10

It was previously demonstrated that two isoforms of HGF (i.e., flHGF and dHGF) can be generated by alternative splicing between exon 4 and exon 5 from each of the constructs. In addition, among the various HGF constructs, HGF-X7 showed the highest level of expression of two isoforms of HGF (i.e., flHGF and dHGF) as disclosed in U.S. Pat. No. 7,812,146, incorporated by reference in its entirety herein. Accordingly, a nucleic acid construct comprising HGF-X7 can be used in preferred embodiments of the methods of the present invention.

In a particularly preferred embodiment, pCK-HGF-X7 (also called “VM202”) (SEQ ID NO:11) is used in the methods described herein. pCK-HGF-X7 was deposited under the terms of the Budapest Treaty at the Korean Culture Center of Microorganisms (KCCM) under accession number KCCM-10361 on Mar. 12, 2002.

The amino acid sequences and nucleotide sequences of HGF isoforms used in the methods described herein may further include amino acid sequences and nucleotide sequences substantially identical to sequences of the wild type human HGF isoforms. The substantial identity includes sequences with at least 80% identity, more preferably at least 90% identity and most preferably at least 95% identity where the amino acid sequence or nucleotide sequence of the wild type human HGF isoform is aligned with a sequence in the maximal manner. Methods of alignment of sequences for comparison are well-known in the art. Specifically, alignment algorithm disclosed in the NCBI Basic Local Alignment Search Tool (BLAST) of the National Center for Biological Information (NBC1, Bethesda, Md.) website and used in connection with the sequence analysis programs blastp, blasm, blastx, tblastn and tblastx can be used to determine the percent identity.

6.3.5.2. Vector

Constructs used in the methods of the present invention typically comprise a vector with one or more regulatory sequences (e.g., a promoter or an enhancer) operatively linked to the expressed sequences. The regulatory sequence regulates expression of the isoforms of HGF.

It is preferred that the polynucleotide encoding one or more isoforms of HGF proteins is operatively linked to a promoter in an expression construct. The term “operatively linked” refers to functional linkage between a nucleic acid expression control sequence (such as a promoter, signal sequence, or array of transcription factor binding sites) and a second nucleic acid sequence, wherein the expression control sequence affects transcription and/or translation of the nucleic acid corresponding to the second sequence.

In typical embodiments, the promoter linked to the polynucleotide is operable in, preferably, animal, more preferably, mammalian cells, to control transcription of the polynucleotide, including the promoters derived from the genome of mammalian cells or from mammalian viruses, for example, CMV (cytomegalovirus) promoter, the adenovirus late promoter, the vaccinia virus 7.5K promoter, SV40 promoter, HSV tk promoter, RSV promoter, EF1 alpha promoter, metallothionein promoter, beta-actin promoter, human IL-2 gene promoter, human IFN gene promoter, human IL-4 gene promoter, human lymphotoxin gene promoter and human GM-CSF gene promoter, but not limited to. More preferably, the promoter useful in this invention is a promoter derived from the IE (immediately early) gene of human CMV (hCMV) or EF1 alpha promoter, most preferably hCMV IE gene-derived promoter/enhancer and 5′-UTR (untranslated region) comprising the overall sequence of exon 1 and exon 2 sequence spanning a sequence immediately before the ATG start codon.

The expression cassette used in this invention may comprise a polyadenylation sequence, for example, including bovine growth hormone terminator (Gimmi, E. R., et al., Nucleic Acids Res. 17:6983-6998 (1989)), SV40-derived polyadenylation sequence (Schek, N, et al., Mol. Cell Biol. 12:5386-5393 (1992)), HIV-1 polyA (Klasens, B. I. F., et al., Nucleic Acids Res. 26:1870-1876 (1998)), β-globin polyA (Gil, A., et al, Cell 49:399-406 (1987)), HSV TK polyA (Cole, C. N. and T. P. Stacy, Mol. Cell. 5 Biol. 5: 2104-2113 (1985)) or polyoma virus polyA (Batt, D. Band G. G. Carmichael, Mol. Cell. Biol. 15:4783-4790 (1995)), but not limited to.

6.3.5.2.1. Non-Viral Vector

In some embodiments, the nucleic acid construct is a non-viral vector capable of expressing two or more isoforms of HGF.

In typical embodiments, the non-viral vector is a plasmid. In currently preferred embodiments, the plasmid is pCK, pCP, pVAXl or pCY. In particularly preferred embodiments, the plasmid is pCK, details of which can be found in WO 2000/040737 and Lee et al., Biochem. Biophys. Res. Comm. 272:230-235 (2000), both of which are incorporated herein by reference in their entireties. E. coli transformed with pCK (Top10-pCK) was deposited at the Korean Culture Center of Microorganisms (KCCM) under the terms of the Budapest Treaty on Mar. 21, 2003 (Accession NO: KCCM-10476). E. coli transformed with pCK-VEGF165 (i.e., pCK vector with VEGF coding sequence—Top10-pCK/VEGF165′) was deposited at the Korean Culture Center of Microorganisms (KCCM) under the terms of the Budapest Treaty on Dec. 27, 1999 (Accession NO: KCCM-10179).

The pCK vector is constructed such that the expression of a gene, e.g., an HGF gene, is regulated under enhancer/promoter of the human cytomegalovirus (HCMV), as disclosed in detail in Lee et al., Biochem. Biophys. Res. Commun. 272: 230 (2000); WO 2000/040737, both of which are incorporated by reference in their entirety. pCK vector has been used for clinical trials on human body, and its safety and efficacy were confirmed (Henry et al., Gene Ther. 18:788 (2011)).

In particularly preferred embodiments, the pCK plasmid containing the HGF-X7 expression sequences is used as the nucleic acid construct in the methods of the present invention. One preferred embodiment, pCK-HGF-X7 (also called “VM202”), has been deposited (in the form of an E. coli strain transformed with the plasmid) under the terms of the Budapest Treaty at the KCCM under accession number KCCM-10361.

6.3.5.2.2. Viral Vector

In other embodiments, various viral vectors known in the art can be used to deliver and express one or more isoforms of HGF proteins of the present invention. For example, vectors developed using retroviruses, lentiviruses, adenoviruses, or adeno-associated viruses can be used for some embodiments of the present invention.

(a) Retrovirus

Retroviruses capable of carrying relatively large exogenous genes have been used as viral gene delivery vectors in the senses that they integrate their genome into a host genome and have broad host spectrum.

In order to construct a retroviral vector, the polynucleotide of the invention is inserted into the viral genome in the place of certain viral sequences to produce a replication-defective virus. To produce virions, a packaging cell line containing the gag, pol and env genes but without the LTR (long terminal repeat) and W components is constructed (Mann et al., Cell, 33:153-159(1983)). When a recombinant plasmid containing the polynucleotide of the invention, LTR and W is introduced into this cell line, the W sequence allows the RNA transcript of the recombinant plasmid to be packaged into viral particles, which are then secreted into the culture media (Nicolas and Rubinstein “Retroviral vectors,” In: Vectors: A survey of molecular cloning vectors and their uses, Rodriguez and Denhardt (eds.), Stoneham: Butterworth, 494-513(1988)) The media containing the recombinant retroviruses is then collected, optionally concentrated and used for gene delivery.

A successful gene transfer using the second generation retroviral vector has been reported. Kasahara et al. (Science, 266:1373-1376 (1994)) prepared variants of moloney murine leukemia virus in which the EPO (erythropoietin) sequence is inserted in the place of the envelope region, consequently, producing chimeric proteins having novel binding properties. Likely, the present gene delivery system can be constructed in accordance with the construction strategies for the second-generation retroviral vector.

(b) Lentiviruses

Lentiviruses can be also used in some embodiments of the present invention. Lentiviruses are a subclass of Retroviruses. However, Lentivirus can integrate into the genome of non-dividing cells, while Retroviruses can infect only dividing cells.

Lentiviral vectors are usually produced from packaging cell line, commonly HEK293, transformed with several plasmids. The plasmids include (1) packaging plasmids encoding the virion proteins such as capsid and the reverse transcriptase, (2) a plasmid comprising an exogenous gene to be delivered to the target.

When the virus enters the cell, the viral genome in the form of RNA is reverse-transcribed to produce DNA, which is then inserted into the genome by the viral integrase enzyme. Thus, the exogenous delivered with the Lentiviral vector can remain in the genome and is passed on to the progeny of the cell when it divides.

(c) Adenovirus

Adenovirus has been usually employed as a gene delivery system because of its mid-sized genome, ease of manipulation, high titer, wide target-cell range, and high infectivity. Both ends of the viral genome contains 100-200 bp ITRs (inverted terminal repeats), which are cis elements necessary for viral DNA replication and packaging. The E1 region (E1A and E1B) encodes proteins responsible for the regulation of transcription of the viral genome and a few cellular genes. The expression of the E2 region (E2A and E2B) results in the synthesis of the proteins for viral DNA replication.

Of adenoviral vectors developed so far, the replication incompetent adenovirus having the deleted E1 region is usually used. The deleted E3 region in adenoviral vectors may provide an insertion site for transgenes (Thimmappaya, B. et al., Cell, 31:543-551(1982); and Riordan, J. R. et al., Science, 245:1066-1073 (1989)). Therefore, it is preferred that the decorin-encoding nucleotide sequence is inserted into either the deleted E1 region (E1A region and/or E1B 5 region, preferably, E1B region) or the deleted E3 region. The polynucleotide of the invention may be inserted into the deleted E4 region. The term “deletion” with reference to viral genome sequences encompasses whole deletion and partial deletion as well. In nature, adenovirus can package approximately 105% of the wildtype genome, providing capacity for about 2 extra kb of DNA (Ghosh-Choudhury et al., EMBO J.′ 6:1733-1739 (1987)). In this regard, the foreign sequences described above inserted into adenovirus may be further 15 inserted into adenoviral wild-type genome.

The adenovirus may be of any of the known serotypes or subgroups A-F. Adenovirus type 5 of subgroup C is the most preferred starting material for constructing the adenoviral gene delivery system of this invention. A great deal of biochemical and genetic information about adenovirus type 5 is known. The foreign genes delivered by the adenoviral gene delivery system are episomal, and genotoxicity to host cells. Therefore, gene therapy using the adenoviral gene delivery system may be considerably safe.

(d) Adeno-Associated Virus (AAV)

Adeno-associated viruses are capable of infecting non-dividing cells and various types of cells, making them useful in constructing the gene delivery system of this invention. The detailed descriptions for use and preparation of AAV vector are found in U.S. Pat. Nos. 5,139,941 and 4,797,368.

Research results for AAV as gene delivery systems are disclosed in LaFace et al, Viology, 162: 483486 (1988), Zhou et al., Exp. Hematol. (NY), 21:928-933(1993), Walsh et al, J. Clin. Invest., 94:1440-1448(1994) and Flotte et al., Gene Therapy, 2:29-37(1995). Typically, a recombinant AAV virus is made by cotransfecting a plasmid containing the gene of interest (i.e., nucleotide sequence of interest to be delivered) flanked by the two AAV terminal repeats (McLaughlin et al., 1988; Samulski et al., 1989) and an expression plasmid containing the wild type AAV coding sequences without the terminal repeats (McCarty et al., J. Viral., 65:2936-2945(1991)).

(e) Other Viral Vectors

Other viral vectors may be employed as a gene delivery system in the present invention. Vectors derived from viruses such as vaccinia virus (Puhlmann M. et al., Human Gene Therapy 10:649-657(1999); Ridgeway, “Mammalian expression vectors,” In: Vectors: A survey of molecular cloning vectors and their uses. Rodriguez and Denhardt, eds. Stoneham: Butterworth, 467-492 (1988); Baichwal and Sugden, “Vectors for gene transfer derived from animal DNA viruses: Transient and stable expression of transferred genes,” In: Kucherlapati R, ed. Gene transfer. New York: Plenum Press, 117-148 (1986) and Coupar et al., Gene, 68:1-10(1988)), lentivirus (Wang G. et al., J. Clin. Invest. 104 (11): RS 5-62 (1999)) and herpes simplex virus (Chamber R., et al., Proc. Natl. 10 15 Acad. Sci USA 92:1411-1415(1995)) may be used in the present delivery systems for transferring both the polynucleotide of the invention into cells.

6.3.6. Administration of Nucleic Acid Construct Expressing Two Hepatocyte Growth Factor (HGF) Isoforms

6.3.6.1. Delivery Methods

Various delivery methods can be used to administer the polynucleotide construct expressing one or more isoforms of HGF in the methods described herein.

6.3.6.1.1. Injection

In typical embodiments, the nucleic acid construct is administered by injection of a liquid pharmaceutical composition.

In currently preferred embodiments, the polynucleotide construct is administered by intramuscular injection. Typically, the polynucleotide construct is administered by intramuscular injection close to the site of pain or patient-perceived site of pain. In some embodiments, the polynucleotide constructs are administered to the muscles of hands, feet, legs, or arms of the subject.

In some embodiments, the construct is injected subcutaneously or intradermally.

In some embodiments, the polynucleotide construct is administered by intravascular delivery. In certain embodiments, the construct is injected by retrograde intravenous injection.

6.3.6.1.2. Electroporation

Transformation efficiency of plasmid DNA into cells in vivo can in some instances be improved by performing injection followed by electroporation. Thus, in some embodiments, the polynucleotide is administered by injection followed by electroporation. In particular embodiments, electroporation is administered using the TriGrid™ Delivery System (Ichor Medical Systems, Inc., San Diego, USA).

6.3.6.1.3. Sonoporation

In some embodiments, sonoporation is used to enhance transformation efficiency of a construct of the present invention. Sonoporation utilizes ultrasound wave to temporarily permeabilize the cell membrane to allow cellular uptake of DNA. Polynucleotide constructs can be incorporated within microbubbles and administered into systemic circulation, followed by external application of ultrasound. The ultrasound induces cavitation of the microbubble within the target tissue to result in release and transfection of the constructs.

6.3.6.1.4. Magnetofection

In some embodiments, magnetofection is used to enhance transformation efficiency of a construct of the present invention. The construct is administered after being coupled to a magnetic nanoparticle. Application of high gradient external magnets cause the complex to be captured and held at the target. The polynucleotide construct can be released by enzymatic cleavage of cross linking molecule, charge interaction or degradation of the matrix.

6.3.6.1.5. Liposome

In some embodiments, polynucleotide of the present invention can be delivered by liposomes. Liposomes are formed spontaneously when phospholipids are suspended in an excess of aqueous medium. Liposome-mediated nucleic acid delivery has been very successful as described in Nicolau and Sene, Biochim. Biophys. Acta, 721:185-190(1982) and Nicolau et al., Methods Enzymol., 149:157-176 (1987). Example of commercially accessible reagents for transfecting animal cells using liposomes includes Lipofectamine (Gibco BRL). Liposomes entrapping polynucleotide of the invention interact with cells by mechanism such as endocytosis, adsorption and fusion and then transfer the sequences into cells.

6.3.6.1.6. Transfection

When a viral vector is used to deliver a polynucleotide encoding HGF, the polynucleotide sequence may be delivered into cells by various viral infection methods known in the art. The infection of host cells using viral vectors are described in the above-mentioned cited documents.

Preferably, the pharmaceutical composition of this invention may be administered parenterally. For non-oral administration, intravenous injection, intraperitoneal injection, intramuscular injection, subcutaneous injection, or local injection may be employed. For example, the pharmaceutical composition may be injected by retrograde intravenous injection.

Preferably, the pharmaceutical composition of the present invention may be administered into the muscle. In some embodiments, the administration is targeted to the muscle affected by the neuropathic pain.

6.3.6.2. Dose

The polynucleotide construct is administered in a therapeutically effective dose. In the methods described herein, the therapeutically effective dose is a dose effective to treat neuropathy in the subject.

In some embodiments of the methods described herein, the polynucleotide construct is administered at a total dose of 1 μg to 200 mg, 1 mg to 200 mg, 1 mg to 100 mg, 1 mg to 50 mg, 1 mg to 20 mg, 5 mg to 10 mg, 16 mg, 8 mg, or 4 mg.

In typical embodiments, the total dose is divided into a plurality of individual injection doses. In some embodiments, the total dose is divided into a plurality of equal injection doses. In some embodiments, the total dose is divided into unequal injection doses.

In various divided dose embodiments, the total dose is administered to 4, 8, 16, 24, or 32 different injection sites.

In some embodiments, the injection dose is between 0.1-5 mg. In certain embodiments, the injection dose is 0.1 mg, 0.15 mg, 0.2 mg, 0.25 mg, 0.3 mg, 0.35 mg, 0.4 mg, 0.45 mg, or 0.5 mg.

The total dose can be administered during one visit or over two or more visits.

In typical divided dose embodiments, all of the plurality of injection doses are administered within 1 hour of one another. In some embodiments, all of the plurality of injection doses are administered within 1.5, 2, 2.5 or 3 hours of one another.

In various embodiments of the methods, a total dose of polynucleotide construct, whether administered as a single unitary dose or divided into plurality of injection doses, is administered only once to the subject.

In some embodiments, administration of a total dose of polynucleotide construct into a plurality of injection sites over one, two, three or four visits can comprise a single cycle. In particular, administration of 32 mg, 16 mg, 8 mg, or 4 mg of polynucleotide construct into a plurality of injection sites over two visits can comprise a single cycle. The two visits can be 3, 5, 7, 14, 21 or 28 days apart.

In some embodiments, the cycle can be repeated. The cycle can be repeated twice, three times, four times, five times, six times, or more.

In some embodiments, the cycle can be repeated 1, 2, 3, 4, 5, 6, 7, 8, 9, 10, 11, 12, or more months after the previous cycle.

In some embodiments, the total dose administered in the subsequent cycle is same as the total dose administered in the prior cycle. In some embodiments, the total dose administered in the subsequent cycle is different from the total dose administered in the prior cycle.

In currently preferred embodiments, the nucleic acid construct is administered at a dose of 8 mg per affected limb, equally divided into a plurality of intramuscular injections and plurality of visits, wherein each of the plurality of injections in any single visit is performed at a separate injection site. In certain embodiments, the nucleic acid construct is administered at a dose of 8 mg per affected limb, equally divided into a first dose of 4 mg per limb on day 0 and a second dose of 4 mg per limb on day 14, wherein each of the first and second dose is equally divided into a plurality of injection doses.

The actual amount administered, and rate and time-course of administration, will depend on the nature and severity of neuropathy being treated. In typical embodiments, the polynucleotide construct is administered in an amount effective to reduce symptoms of neuropathy, for example, neuropathic pain. In some embodiments, the amount is effective to reduce neuropathic pain within 1 week of administration. In some embodiments, the amount is effective to reduce neuropathic pain within 2 weeks, 3 weeks, or 4 weeks of administration.

In some emb are bigger>>and<<odiments, two different types of constructs are administered together to induce expression of two isoforms of HGF, i.e., a first construct encoding flHGF and a second construct encoding dHGF. In some embodiments, a single construct that encodes both flHGF and dHGF is delivered to induce expression of both flHGF and dHGF.

According to the conventional techniques known to those skilled in the art, the pharmaceutical composition may be formulated with pharmaceutically acceptable carrier and/or vehicle as described above, finally providing several forms a unit dose form and a multidose form. Non-limiting examples of the formulations include, but not limited to, a solution, a suspension or an emulsion in oil or aqueous medium, an extract, an elixir, a powder, a granule, a tablet and a capsule, and may further comprise a dispersion agent or a stabilizer.

6.3.6.3. Variations

In vivo and/or in vitro assays may optionally be employed to help identify optimal dosage ranges. The precise dose to be employed in the formulation will also depend on the route of administration, and the seriousness of the condition, and should be decided according to the judgment of the practitioner and each subject's circumstances. Effective doses may be extrapolated from dose-response curves derived from in vitro or animal model test systems.

The polynucleotide construct can be administered alone or in combination with other treatments, either simultaneously or sequentially dependent upon the condition to be treated.

### 6.4. Pharmaceutical Compositions

In typical embodiments, the nucleic acid construct is administered in a liquid pharmaceutical composition.

6.4.1. Pharmacological Compositions and Unit Dosage Forms Adapted for Injection

For intravenous, intramuscular, intradermal, or subcutaneous injection, the nucleic acid construct will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required.

In various embodiments, the nucleic acid construct is present in the liquid composition at a concentration of 0.01 mg/ml, 0.05 mg/ml, 0.1 mg/ml, 0.25 mg/ml, 0.5 mg/ml, or 1 mg/ml. In some embodiments, the unit dosage form is a vial containing 2 ml of the pharmaceutical composition at a concentration of 0.01 mg/ml, 0.1 mg/ml, 0.5 mg/ml, or 1 mg/ml.

In some embodiments, the unit dosage form is a vial, ampule, bottle, or pre-filled syringe. In some embodiments, the unit dosage form contains 0.01 mg, 0.1 mg, 0.2 mg, 0.25 mg, 0.5 mg, 1 mg, 2.5 mg, 5 mg, 8 mg, 10 mg, 12.5 mg, 16 mg, 24 mg, 25 mg, 50 mg, 75 mg, 100 mg, 150 mg, or 200 mg of the polynucleotide of the present invention.

In typical embodiments, the pharmaceutical composition in the unit dosage form is in liquid form. In various embodiments, the unit dosage form contains between 0.1 mL and 50 ml of the pharmaceutical composition. In some embodiments, the unit dosage form contains 0.25 ml, 0.5 ml, 1 ml, 2.5 ml, 5 ml, 7.5 ml, 10 ml, 25 ml, or 50 ml of pharmaceutical composition.

In particular embodiments, the unit dosage form is a vial containing 1 ml of the pharmaceutical composition at Unit dosage form embodiments suitable for subcutaneous, intradermal, or intramuscular administration include preloaded syringes, auto-injectors, and auto-inject pens, each containing a predetermined amount of the pharmaceutical composition described hereinabove.

In various embodiments, the unit dosage form is a preloaded syringe, comprising a syringe and a predetermined amount of the pharmaceutical composition. In certain preloaded syringe embodiments, the syringe is adapted for subcutaneous administration. In certain embodiments, the syringe is suitable for self-administration. In particular embodiments, the preloaded syringe is a single use syringe.

In various embodiments, the preloaded syringe contains about 0.1 mL to about 0.5 mL of the pharmaceutical composition. In certain embodiments, the syringe contains about 0.5 mL of the pharmaceutical composition. In specific embodiments, the syringe contains about 1.0 mL of the pharmaceutical composition. In particular embodiments, the syringe contains about 2.0 mL of the pharmaceutical composition.

In certain embodiments, the unit dosage form is an auto-inject pen. The auto-inject pen comprises an auto-inject pen containing a pharmaceutical composition as described herein. In some embodiments, the auto-inject pen delivers a predetermined volume of pharmaceutical composition. In other embodiments, the auto-inject pen is configured to deliver a volume of pharmaceutical composition set by the user.

In various embodiments, the auto-inject pen contains about 0.1 mL to about 5.0 mL of the pharmaceutical composition. In specific embodiments, the auto-inject pen contains about 0.5 mL of the pharmaceutical composition. In particular embodiments, the auto-inject pen contains about 1.0 mL of the pharmaceutical composition. In other embodiments, the auto-inject pen contains about 5.0 mL of the pharmaceutical composition.

6.4.2. Lyophilized DNA Formulations

In some embodiments, nucleic acid constructs of the present inventions are administered as liquid compositions reconstituted from lyophilized formulations. In specific embodiments, DNA formulations lyophilized as disclosed in U.S. Pat. No. 8,389,492, incorporated by reference in its entirety herein, are used after reconstitution.

In some embodiments, the nucleic acid constructs of the present invention is formulated with certain excipients, including a carbohydrate and a salt, prior to lyophilization. The stability of a lyophilized formulation of DNA to be utilized as a diagnostic or therapeutic agent can be increased by formulating the DNA prior to lyophilization with an aqueous solution comprising a stabilizing amount of carbohydrate.

A carbohydrate of the DNA formulation of the invention is a mono-, oligo-, or polysaccharide, such as sucrose, glucose, lactose, trehalose, arabinose, pentose, ribose, xylose, galactose, hexose, idose, mannose, talose, heptose, fructose, gluconic acid, sorbitol, mannitol, methyl a-glucopyranoside, maltose, isoascorbic acid, ascorbic acid, lactone, sorbose, glucaric acid, erythrose, threose, allose, altrose, gulose, erythrulose, ribulose, xylulose, psicose, tagatose, glucuronic acid, galacturonic acid, mannuronic acid, glucosamine, galactosamine, neuraminic acid, arabinans, fructans, fucans, galactans, galacturonans, glucans, mannans, xylans, levan, fucoidan, carrageenan, galactocarolose, pectins, pectic acids, amylose, pullulan, glycogen, amylopectin, cellulose, dextran, cyclodextrin, pustulan, chitin, agarose, keratin, chondroitin, dermatan, hyaluronic acid, alginic acid, xantham gum, or starch.

In one series of embodiments, the carbohydrate is mannitol or sucrose.

The carbohydrate solution prior to lyophilization can correspond to carbohydrate in water alone, or a buffer can be included. Examples of such buffers include PBS, HEPES, TRIS or TRIS/EDTA. Typically the carbohydrate solution is combined with the DNA to a final concentration of about 0.05% to about 30% sucrose, typically 0.1% to about 15% sucrose, such as 0.2% to about 5%, 10% or 15% sucrose, preferably between about 0.5% to 10% sucrose, 1% to 5% sucrose, 1% to 3% sucrose, and most preferably about 1.1% sucrose.

A salt of the DNA formulation of the invention is NaCl or KCl. In certain aspects, the salt is NaCl. In further aspects, the salt of the DNA formulation is in an amount selected from the group consisting of between about 0.001% to about 10%, between about 0.1% and 5%, between about 0.1% and 4%, between about 0.5% and 2%, between about 0.8% and 1.5%, between about 0.8% and 1.2% w/v. In certain embodiments, the salt of the DNA formulation is in an amount of about 0.9% w/v.

The final concentration in liquid compositions reconstituted from lyophilized formulations is from about 1 ng/mL to about 30 mg/mL of plasmid. For example, a formulation of the present invention may have a final concentration of about 1 ng/mL, about 5 ng/mL, about 10 ng/mL, about 50 ng/mL, about 100 ng/mL, about 200 ng/mL, about 500 ng/mL, about 1 μg/mL, about 5 μg/mL, about 10 μg/mL, about 50 μg/mL, about 100m/mL, about 200 μg/mL, about 400m/mL, about 500m/mL, about 600m/mL, about 800m/mL, about 1 mg/mL, about 2 mg/mL, about 2.5 mg/mL, about 3 mg/mL, about 3.5 mg/mL, about 4 mg/mL, about 4.5 mg/mL, about 5 mg/mL, about 5.5 mg/mL, about 6 mg/mL, about 7 mg/mL, about 8 mg/mL, about 9 mg/mL, about 10 mg/mL, about 20 mg/mL, or about 30 mg mg/mL of a plasmid. In certain embodiments of the invention, the final concentration of the DNA is from about 100 μm/mL to about 2.5 mg/mL. In particular embodiments of the invention, the final concentration of the DNA is from about 0.5 mg/mL to 1 mg/mL.

The DNA formulation of the invention is lyophilized under standard conditions known in the art. A method for lyophilization of the DNA formulation of the invention may comprise (a) loading a container, e.g., a vial, with a DNA formulation, e.g., a DNA formulation comprising a plasmid DNA, a salt and a carbohydrate, where the plasmid DNA comprises an HGF gene, or variant thereof, into a lyophilizer, wherein the lyophilizer has a starting temperature of about 5° C. to about −50° C.; (b) cooling the DNA formulation to subzero temperatures (e.g., −10° C. to −50° C.); and (c) substantially drying the DNA formulation. The conditions for lyophilization, e.g., temperature and duration, of the DNA formulation of the invention can be adjusted by a person of ordinary skill in the art taking into consideration factors that affect lyophilization parameters, e.g., the type of lyophilization machine used, the amount of DNA used, and the size of the container used.

The container holding the lyophilized DNA formulation may then be sealed and stored for an extended period of time at various temperatures (e.g., room temperature to about −180° C., preferably about 2-8° C. to about −80° C., more preferably about −20° C. to about −80° C., and most preferably about −20° C.). In certain aspects, the lyophilized DNA formulations are preferably stable within a range of from about 2-8° C. to about −80° C. for a period of at least 6 months without losing significant activity. Stable storage plasmid DNA formulation can also correspond to storage of plasmid DNA in a stable form for long periods of time before use as such for research or plasmid-based therapy. Storage time may be as long as several months, 1 year, 5 years, 10 years, 15 years, or up to 20 years. Preferably the preparation is stable for a period of at least about 3 years.

### 6.5. Examples

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations can be used, e.g., bp, base pair(s); kb, kilobase(s); pl, picoliter(s); s or sec, second(s); min, minute(s); h or hr, hour(s); aa, amino acid(s); nt, nucleotide(s); and the like.

The practice of the present invention will employ, unless otherwise indicated, conventional methods of protein chemistry, biochemistry, recombinant DNA techniques and pharmacology, within the skill of the art.

### 6.5.1. Example 1: Effects of Gabapentin on VM202-Mediated Pain Reduction in Chronic Constriction Injury (CCI) Animal Model for Neuropathy

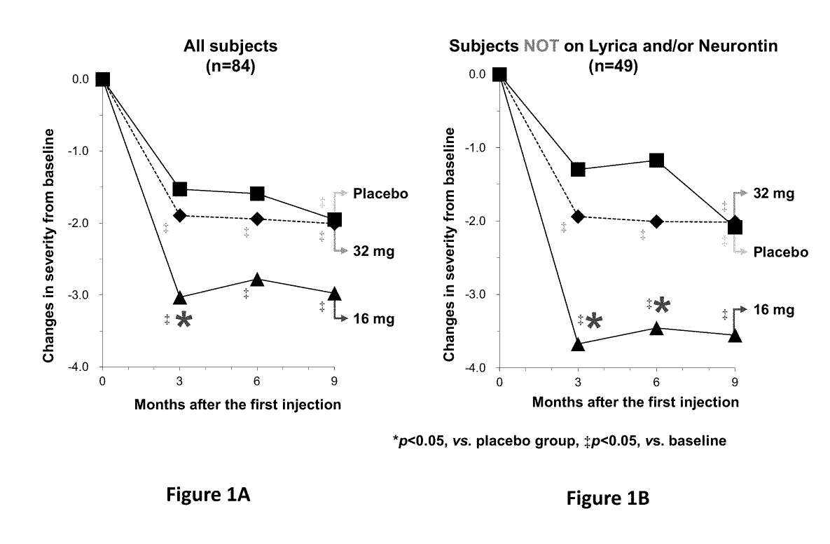

FIG. 1A, reproduced from Kessler et al., Annals Clin. Transl. Neurology 2(5):465-478 (2015), shows time-course change in pain levels measured in all patients in the phase 2 clinical trial of VM202 for treatment of diabetic peripheral neuropathy. The data show pain severity measured at 3, 6, and 9 months after the administration of a high dose of VM202 (8 mg per leg on day 0, administered as a plurality of intramuscular injections; 8 mg per leg on day 14, administered as a plurality of intramuscular injections; total dose across both legs and both visits, 32 mg), a low dose of VM202 (4 mg per leg on day 0, administered as a plurality of intramuscular injections; 4 mg per leg on day 14, administered as a plurality of intramuscular injections; total dose across both legs and both visits, 16 mg), or saline (placebo). FIG. 1B, also reproduced from Kessler et al., Annals Clin. Transl. Neurology 2(5):465-478 (2015), shows time-course change in pain levels measured in a group of patients who were not on Lyrica (pregabalin) and/or Neurontin (gabapentin), 3, 6, and 9 months after administering the high dose of VM202, the low dose of VM202, or saline (placebo). As also reported in Kessler et al., patients who were not on Lyrica and/or Neurontin (FIG. 1B) generally experienced a larger reduction in pain from baselines than the total patient group (FIG. 1A) after administration of the low dose of VM202.

The post hoc analysis of the phase II clinical trial data could not elucidate the physiological mechanism underlying the apparent deleterious interaction of gabapentinoids with VM202. In particular, the data could not predict whether prior administration of a gabapentinoid would preclude later efficacy of VM202, nor predict how to administer VM202 efficaciously to patients who had previously taken gabapentinoids.

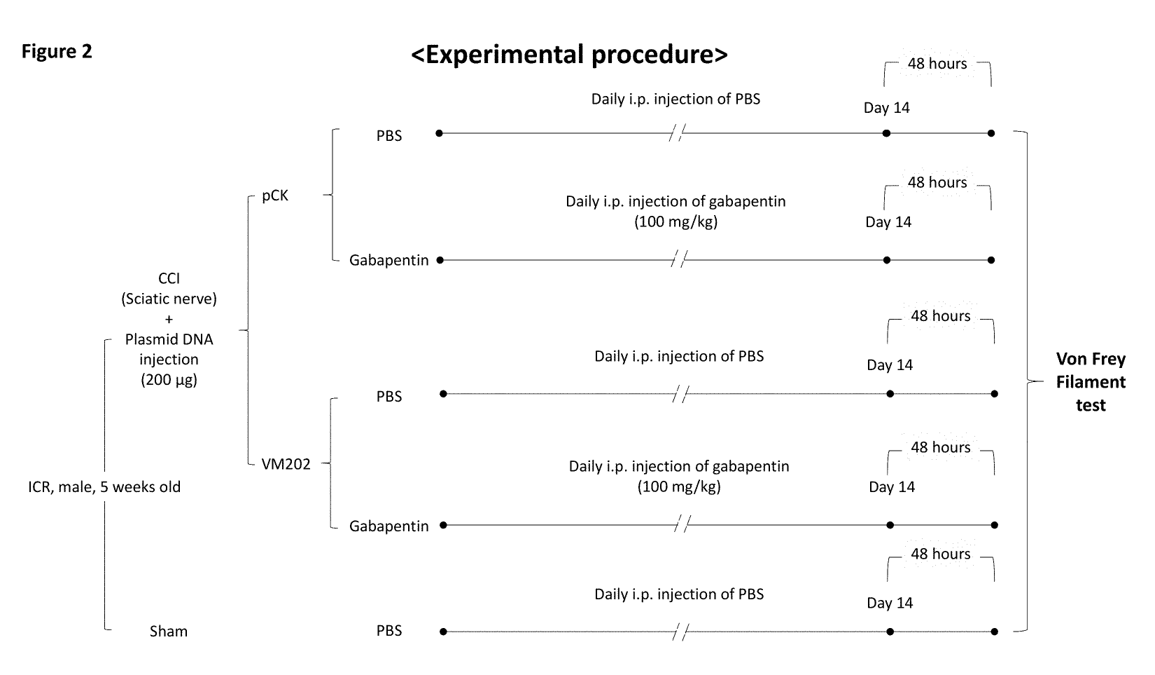

In order to explore the mechanisms behind the gabapentinoid interference with VM202 efficacy, we tested effects of gabapentin on VM202-mediated pain reduction in chronic constriction injury (CCI) mice as presented in FIG. 2. CCI is an animal model widely used for studying neuropathic pain. Specifically, chronic constriction injury (CCI) was introduced by applying loosely constrictive ligatures to the sciatic nerve of 5 week-old male mice. The CCI mice were divided into three groups—in the first group, 200 μg of pCK vector was administered into the cranial thigh muscles as a negative control for VM202 administration (pCK is the vector used in VM202, but lacks the HGF vector payload), in the second group, 200 μg of VM202 was administered into the cranial thigh muscles, and in the third group, no DNA construct was administered. Animals in each of the first and the second group were further divided into two subgroups, with the first subgroup injected daily with 100 mg/kg of gabapentin and the second subgroup injected daily with PBS as a negative control for gabapentin administration, via intraperitoneal cavity for two weeks. The sham group without CCI was also maintained and daily injected with PBS. From day 14 to day 16, Von Frey filament test was performed to assess the level of neuropathic pain (mechanical allodynia).

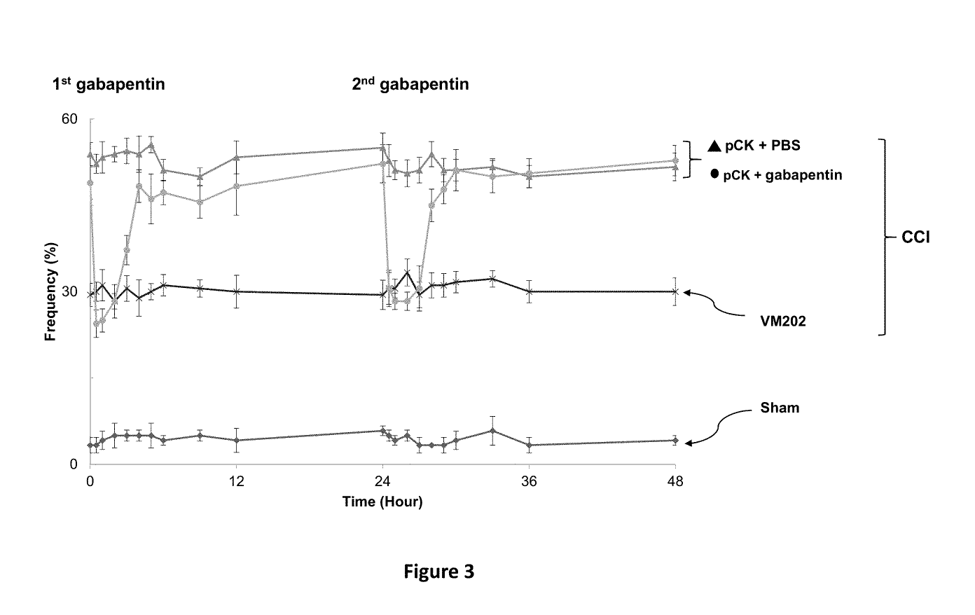

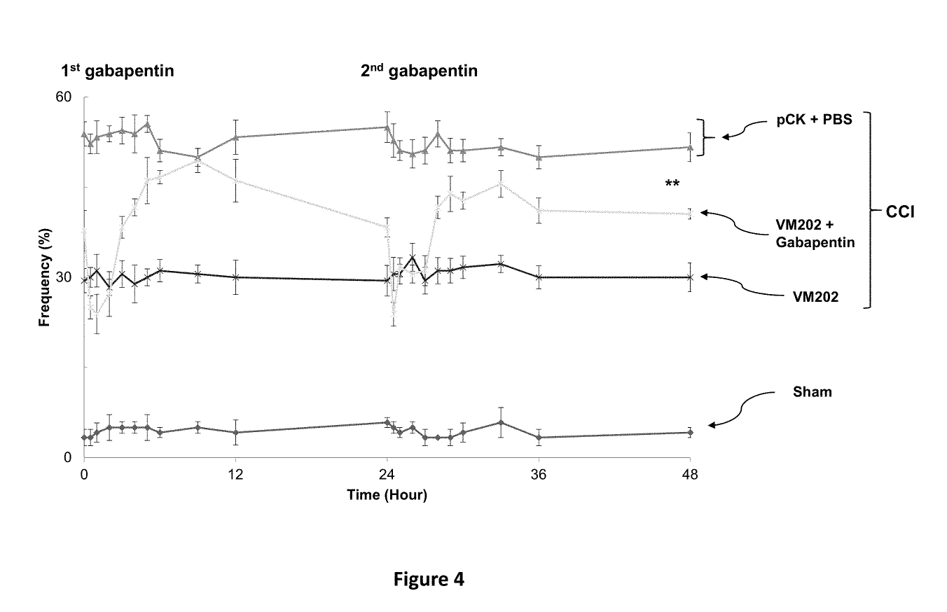

Paw withdrawal frequencies measured by the Von Frey filament test in the five different groups are presented in FIGS. 3 and 4. A sham-operated group (“Sham,” line with diamonds in FIGS. 3 and 4) showed a very low basal frequency of paw withdrawal throughout the experimental period. A CCI-operated group administered pCK (negative control for VM202) and daily injected with PBS (negative control for gabapentin), on the other hand, had continuously high pain level throughout the experimental period (“pCK-PBS,” line with triangles in FIGS. 3 and 4). A CCI-operated group administered pCK and daily injected with gabapentin, on the other hand, showed decreases in pain levels immediately after the gabapentin administration as demonstrated by the reduction in paw withdrawal frequencies (“pCK-Gabapentin,” line with circles in FIG. 3). However, pain relieving effects of gabapentin lasted only for about 6 hours.

A CCI-operated group injected with VM202 showed significantly lower pain levels throughout the experimental period (“VM202,” line with x in FIG. 3). When gabapentin was daily injected to VM202-treated CCI mice (“VM202+ gabapentin,” line with x in FIG. 4), pain level decreased even further from the level achieved by VM202 for a very short time, then the pain level increased for about 10 hours close to the pCK-PBS level, followed by a gradual decrease until the second administration of gabapentin. When gabapentin was administered for the second time 24 hours after the initial injection, the pain level went down again below the VM202 level for a very short time, then rose to a stable point between the pCK-PBS and VM202 levels. Overall, the pain reduction effect of VM202 was compromised by gabapentin by more than 50%, from the frequency of 30.56% to the frequency of 46.1%.

### 6.5.2. Example 2: Effects of Gabapentin on VM202-Mediated Nerve-Regeneration in a Nerve Crush Animal Model for Neuropathy

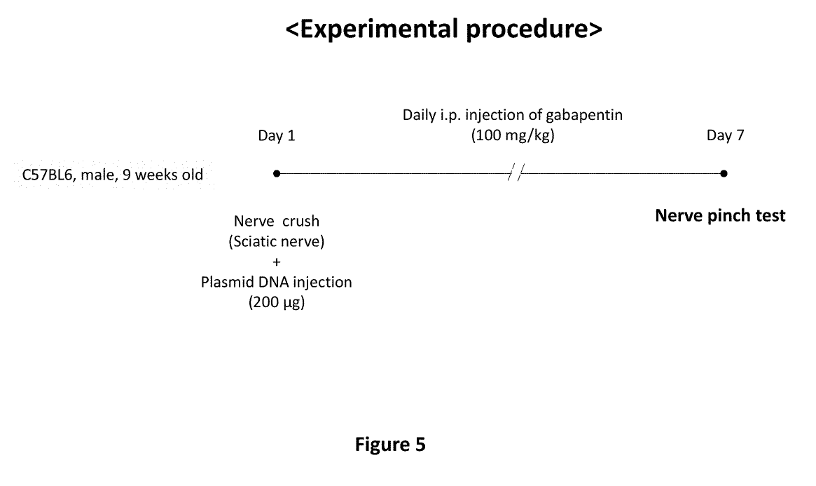

Effects of gabapentinoids on VM202-mediated nerve regeneration were tested in a nerve crush animal model. The protocol is schematized in FIG. 5. Specifically, nerve crush was introduced to 9-week-old C57BL/6 mice by giving brief pressure to their sciatic nerve. On the same day (day 1), the mice were injected with 200 μg of VM202 to the cranial thigh muscles right after the nerve crush. From the next day (day 2), 100 mg/kg gabapentin was administered daily.

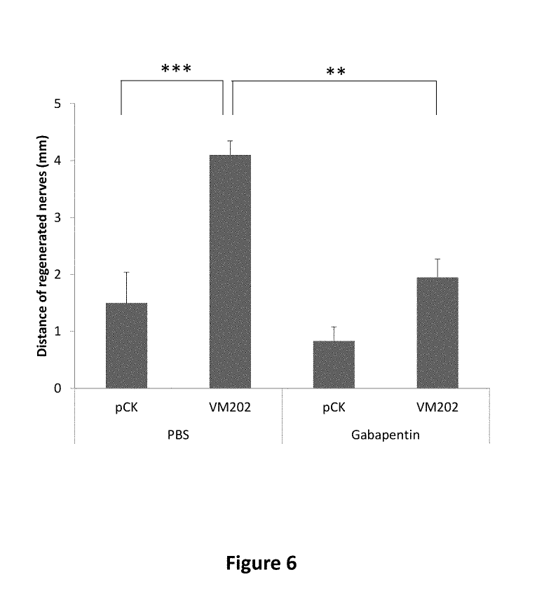

On day 7, a nerve pinch test was performed to quantify functional recovery of the injured nerve. For the nerve pinch test, light anesthesia was induced and sciatic nerve was exposed. The injured nerve was pinched from its distal to proximal direction until a reflex response was observed. The distance was then measured between the injury site and the foremost site that produced the response. The distance measured by this method represents the length of regenerated nerves, which is provided on the y-axis in FIG. 6.

The length of regenerated nerve measured in VM202-treated mice was about 4±0.2 mm, approximately 2.7-fold longer than the length measured in control mice treated with the negative control plasmid vector, pCK (1.5±0.5 mm). This result confirms that VM202 is effective in inducing regeneration of damaged neurons (left two bars labeled “PBS” in FIG. 6). This VM202-mediated enhancement of the nerve regeneration was highly reduced in the mice treated with gabapentin. The length of regenerated nerve measured in VM202-treated mice was 1.95±0.3 mm when daily injected with gabapentin. Thus, nerve regeneration in the mice (1.95±0.3 mm) was significantly less than in mice similarly administered VM202 but without daily injection of gabapentin (4±0.2 mm). This result suggested that gabapentin interfered with VM202-mediated nerve regeneration. Even in the presence of gabapentin, however, VM202 was still able to increase nerve regeneration by 2.3-fold compared to the pCK control group (right two bars labeled “Gabapentin” in FIG. 6).

### 6.5.3. Example 3: Effect of Gabapentin on VM202-Mediated Upregulation of c-Jun

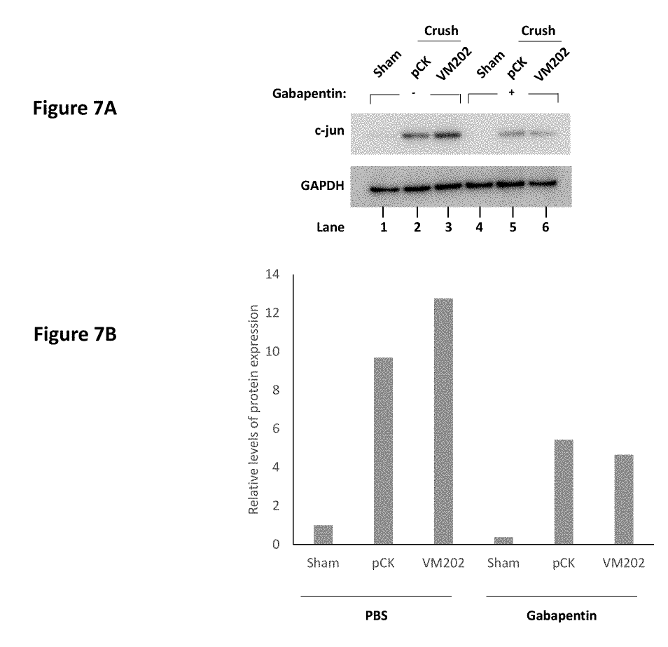

c-Jun is well established to be a key factor involved in nerve regeneration, and has been used as a marker for that process. Expression of c-Jun protein prepared from dorsal root ganglion (DRG) cells obtained from a sham mouse (“Sham”) or from a nerve crush mouse (“Crush”) was measured by Western blot assay, using an antibody against c-Jun. Expression of c-Jun increased significantly in the nerve crush model compared to the sham animal (compare lane 1 and 2 of FIG. 7A). VM202 treatment further increased c-Jun expression level by 1.3-fold (compare lanes 2 and 3 of FIG. 7A), but such induction was not observed when mice were exposed to gabapentin (compare lanes 5 and 6 of FIG. 7A). The western blot assay results were quantified based on the band intensities and presented in FIG. 7B for their analysis and comparison. The data suggested that HGF produced from VM202 might have utilized the calcium signaling pathway to increase the level of c-Jun protein, eventually leading to regeneration of the injured nerve.

### 6.5.4. Example 4: Interference of Therapeutic Effects of VM202 by Gabapentin Administered at Different Time Points

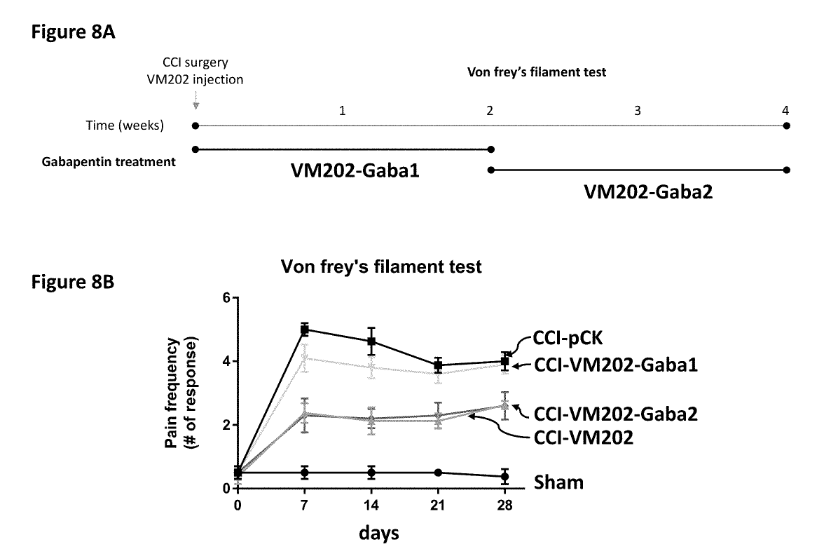

The effects of gabepentinoid administration at different time points relative to VM202 administration were tested in chronic constriction injury (CCI) mice. CCI mice were assigned to five groups and treated as illustrated in FIG. 8A and summarized in Table 1 below.

TABLE 1

| Group | Surgery | VM202 | Gabapentin |
|---|---|---|---|
| Sham | Sham on | No | No |
| | day 0 | | |
| CCI-pCK | CCI on | No (200 μg/head, | No |
| | day 0 | pCK) | |
| CCI-VM202 | CCI on | 200 μg/head VM202 | No |
| | day 0 | i.m. injection on | |
| | | day 0 | |
| CCI-VM202- | CCI on | 200 μg/head VM202 | Gabapentin treatment |
| Gaba1 | day 0 | i.m. injection on | from day 0 to day 14 |
| | | day 0 | (first two weeks) |
| CCI-VM202- | CCI on | 200 μg/head VM202 | Gabapentin treatment |
| Gaba2 | day 0 | i.m. injection on | from day 15 to day 28 |
| | | day 0 | (second two weeks) |

After CCI surgery, the development of mechanical allodynia was assessed using a Von Frey's filament test once a week, and the level of pain reduction fold was calculated based on the mechanical frequency evaluation. Briefly, animals were placed individually in a cylinder on top of a metal mesh floor for adaptation. To examine the frequency of mechanical sensitivity, mice were assessed by stimulating the hind paw using constant thickness of the filament (0.16 g).

Results in FIG. 8B demonstrate that injection of VM202 significantly reduced the pain level (CCI-VM202) compared to control mice injected with pCK vector (CCI-pCK). However, injection of VM202 had no effects, comparable to CCI group treated with pCK vector lacking insert (CCI-pCK), when gabapentin was administered simultaneously with VM202 and daily for the following two weeks (CCI-VM202-Gaba1). This suggests that administration of gabapentin together with and/or shortly after VM202 injection can completely interfere with and abrogate the pain-relieving effects of VM202. Moreover, such interference continued even when there were no additional gabapentin administrations. Specifically, CCI mice treated with VM202 (CCI-VM202-Gaba1) continued to have high level of pains similar to CCI control mice treated with pCK vector (CCI-pCK) from day 14 to 28, when there were no additional gabapentin administrations.

Interference of therapeutic effects of VM202 by gabapentin, however, was not observed when gabapentin treatment was initiated 14 days after VM202 injection (CCI-VM202-Gaba2). When CCI mice were treated with VM202 without gabapentin administration for the first two weeks, the pain relieving effects of VM202 were significant and maintained even when gabapentin was administered later, daily from day 15 to day 28. This suggests that gabapentin does not interfere with therapeutic effects of VM202 when there is sufficient delay between VM202 and gabapentin administrations.

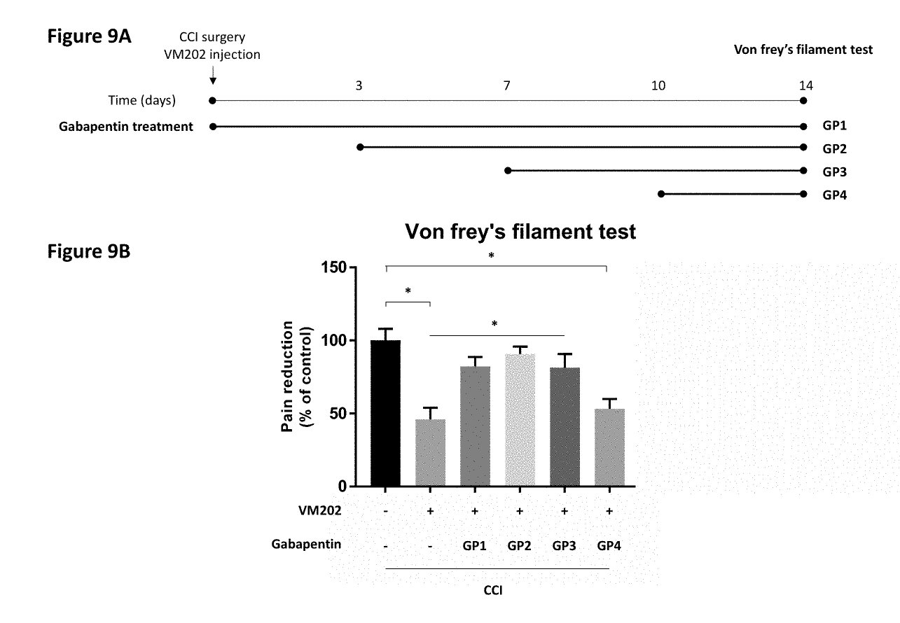

To further understand the delay required to prevent the interference, in a further experiment, CCI mice injected with VM202 were treated with gabapentin after delays for various periods ranging from 0 to 14 days. Specifically, CCI mice were assigned to six groups as illustrated in FIG. 9A and summarized in Table 2 below. CCI mice were injected with VM202 or pCK on day 0. The CCI mice were treated with no gabapentin (CONT1, CONT2) or additionally treated with gabapentin starting on day 0 (on the day of VM202 injection, GP1) or day 3 (GP2), day 7 (GP3) or day 10 (GP4) after VM202 injection.

TABLE 2

| | | | Gabapentin | No. of |
|---|---|---|---|---|
| Group | VM202 | Gabapentin | Initiation | animals |
| CONT1 | 200 μg/head pCK | No | No | 4 |
| CONT2 | 200 μg/head VM202 | No | No | 4 |
| GP1 | 200 μg/head VM202 | 100 mg/kg | Day 0 | 5 |
| GP2 | 200 μg/head VM202 | 100 mg/kg | Day 3 | 4 |
| GP3 | 200 μg/head VM202 | 100 mg/kg | Day 7 | 4 |
| GP4 | 200 μg/head VM202 | 100 mg/kg | Day 10 | 5 |

Two weeks after CCI surgery and VM202 or pCK injection, the development of mechanical allodynia was assessed using a Von Frey's filament test, and the level of pain reduction fold was calculated based on the mechanical frequency evaluation for each animal. The results are provided in FIG. 9B. The results show that VM202 did not have significant pain reducing effects in GP1, GP2, and GP3 whereas VM202 provided significant pain relief in CONT2 or GP4. This suggests that gabapentin treatment together with and/or during the first week of VM202 administration can interfere with therapeutic effects of VM202, but that gabapentin treatment beginning about 10 days after VM202 administration does not have significant effects on the therapeutic efficacy of VM202.

This study suggests that the deleterious effects of gabapentinoids on efficacy and potency of VM202 can be significantly reduced by discontinuing gabapentinoid administration prior to the first dose of VM202, and can be attenuated by withholding gabapentinoid administration for at least about one week after the first dose of VM202.

### 7. INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### 8. EQUIVALENTS

While various specific embodiments have been illustrated and described, the above specification is not restrictive. It will be appreciated that various changes can be made without departing from the spirit and scope of the invention(s). Many variations will become apparent to those skilled in the art upon review of this specification.

What is claimed is:

1. A method of treating neuropathy, comprising the steps of:
selecting a patient with neuropathy who has been administered a gabapentinoid,
discontinuing gabapentinoid administration to the patient, and
administering VM202 to the patient.

2. The method of claim 1, further comprising the step of:
withholding gabapentinoid administration for at least a week after the step of administering VM202.

3. The method of claim 1, further comprising the step of:
withholding gabapentinoid administration for at least 10 days after the step of administering VM202.

4. The method of any of claims 1-3, wherein the step of discontinuing gabapentinoid administration comprises tapering gabapentinoid administration.

5. The method of any of claims 1-4, wherein the step of administering VM202 is performed after a complete cessation of gabapentinoid administration.

6. The method of claim 5, wherein the step of administering VM202 is performed at least 1, 2, 3, 5, 7, 14, 21, 30, 60, or 90 days after a complete cessation of gabapentinoid administration.

7. The method of any of claims 1-6, wherein the neuropathy is diabetic peripheral neuropathy.

8. The method of any of claims 1-6, wherein the neuropathy is post-herpetic neuropathy.

9. The method of any of claims 1-8, wherein the gabapentinoid is gabapentin or pregabalin.

10. The method of any of claims 1-9, wherein the step of administering VM202 comprises administering 8 mg of VM202 per affected limb of the patient, equally divided into a plurality of intramuscular injections and plurality of visits, wherein each of the plurality of intramuscular injections in any single visit is performed at a separate injection site.

11. The method of claim 10, wherein VM202 is administered at a dose of 16 mg equally divided into 64 intramuscular injections,
wherein 16 intramuscular injections are administered to separate injection sites on a first calf on a first visit,
wherein 16 intramuscular injections are administered to separate injection sites on a second calf on the first visit,
wherein 16 intramuscular injections are administered to separate injection sites on the first calf on a second visit,
wherein 16 intramuscular injections are administered to separate injection sites on the second calf on the second visit, and
wherein each of the 64 intramuscular injections is performed with 0.25 mg of VM202 in a volume of 0.5 ml.

12. A method of treating neuropathy by administering VM202, the improvement comprising:
selecting a patient with neuropathy who has been administered a gabapentinoid;
discontinuing gabapentinoid administration to the patient; and then
administering VM202 to the patient.

13. A method of treating neuropathy, comprising the steps of:
determining whether a patient with neuropathy has been administered a gabapentinoid within the preceding week;
if the patient has been administered a gabapentinoid within the preceding week, discontinuing gabapentinoid administration to the patient, and thereafter administering VM202 to the patient; and
if the patient has not been administered a gabapentinoid within the preceding week, administering VM202 to the patient.
